(19) United States
(12) Patent Application Publication
(10) Pub. No.: US 2019/0092761 A1
(43) Pub. Date: Mar. 28, 2019

(54) Methods and Compositions for Inhibition of Bromodomain and Extratermial Proteins

(71) Applicant: Convergene LLC, Gaitherburg, MD (US)

(72) Inventors: Makoto Yoshioka, Gaitherburg, MD (US); Jeffrey William Strovel, Laurel, MD (US); Janak Khimchand Padia, Germantown, MD (US)

(21) Appl. No.: 15/526,609

(22) PCT Filed: Nov. 13, 2015
(86) PCT No.: PCT/US15/60494

§ 371 Date: May 12, 2017

Related U.S. Application Data
(60) Provisional application No. 62/079,005, filed on Nov. 13, 2014.

(51) Int. Cl. C07D 413/04; C07D 401/04; C07D 215/42; C07D 413/14

(52) U.S. Cl. CPC C07D 413/04; C07D 413/14; C07D 215/42; C07D 401/04

(57) ABSTRACT

The present invention relates to compounds that bind to and otherwise modulate the activity of bromodomain-containing proteins, to processes for preparing these compounds, to pharmaceutical compositions containing these compounds, and to methods of using these compounds for treating a wide variety of conditions and disorders.

### FIELD OF THE INVENTION

The present invention relates to compounds that inhibit bromodomain-containing proteins from binding acetylated proteins, to processes for preparing these compounds, to pharmaceutical compositions containing these compounds, and to methods of using these compounds for treating or preventing a wide variety of medical conditions, diseases or disorders.

### BACKGROUND OF THE INVENTION

Epigenetic chromatin remodeling is a central mechanism for the regulation of gene expression. Pharmacological modulation of epigenetic change represents a new mode of therapeutic interventions for cancer and inflammation. Emerging evidence suggests that such epigenetic modulations may also provide therapeutic means for treatment of obesity, as well as metabolic, cardiovascular, neurodegenerative, psychiatric and infectious diseases.

The eukaryotic genome is organized into a basic packaging unit called a nucleosome, which is comprised of approximately 147 base pairs of double-stranded DNA helix wound around a histone octamer, which, in turn, consists of two subunits each of H2A, H2B, H3, and H4 proteins. Nucleosomes are further packaged into chromatin structures, which can exist in a relatively loose state of euchromatin or in a tightly packed heterochromatin structure. Transition from heterochromatin to euchromatin allows transcription of genes, although not all of the genes in euchromatin structure are transcribed. This transition from heterochromatin to euchromatin is controlled by post-translational modifications of histone proteins, including acetylation of lysine residues in H3/H4 proteins. Histone acetylation is catalyzed by histone acetyltransferases (HATs), resulting in open euchromatin structures that allow transcription of genes including tumor suppressor genes. Conversely, histone deacetylation leads to suppression of such genes and this activity is catalyzed by histone deacetylases (HDACs). Inhibition of histone deacetylases is a mode of cancer treatment and vorinostat (Zolinza®), a histone deacetylase inhibitor, has been shown to be an effective drug for cutaneous T-cell lymphoma in humans.

Histone acetylation also is modulated by bromodomain-containing proteins. A bromodomain is an approximately 110 amino acid-long evolutionary conserved bundle of four alpha-helices that binds to acetyl lysine residues of acetylated proteins. These domains are present in a number of chromatin-associated proteins including HATs. Bromodomains were first identified as a novel structural motif in the brahma protein, a regulator of Drosophila homeotic genes, but are also found in proteins in humans and yeast either as single-copy or contiguously repeated domains, and are thought to confer specificity for the complex pattern of epigenetic modifications known as the histone code (Cell. 1992 Feb. 7; 68(3):561-72; J Biomol Screen. 2011 December; 16(10):1170-85). The human genome encodes approximately 50 bromodomain-containing proteins (Bioinformatics. 2004 Jun. 12; 20(9):1416-27), some of which may be involved in etiology of cancer, inflammation, obesity, metabolic, cardiovascular, neurodegenerative, psychiatric and infectious diseases (Med Chem Commun. 2012 Jan. 4 3(2):123-134; Curr Opin Drug Discov Devel. 2009 September; 12(5):659-65; Discov Med. 2010 December; 10(55):489-99; FEBS Lett. 2010 Aug. 4; 584(15):3260-8; J Virol. 2006 September; 80(18):8909-19; J Virol. 2005 July; 79(14):8920-32; Curr Opin Pharmacol. 2008 February; 8(1):57-64). Thus, inhibition and/or modulation of bromodomain-containing proteins may present a new mode of pharmacological intervention for such diseases.

Of approximately 50 bromodomain-containing proteins encoded by the human genome, BET proteins represent a small protein family that includes BRD2, BRD3, BRD4 and BRDT. BET proteins contain two tandem bromodomains followed by an extraterminal (ET) domain for protein-protein interaction in the carboxy-terminal region (J Biol Chem. 2007 May 4; 282(18):13141-5). BET proteins bind to acetylated nucleosomes and are thought to function by opening chromatin structure and/or by facilitating transcriptional initiation (Front Biosci. 2001 Aug. 1; 6:D1008-18).

Previously, inhibition of BRD4, either by a BRD4-specific RNAi or by a small-molecule BET inhibitor (JQ1), was unequivocally shown to induce suppression of MYC oncogene (Nature. 2011 Aug. 3; 478(7370):524-8). This indirect suppression of MYC gene expression as a secondary effect of BRD4 inhibition comprises the central mechanism of action exerted by a BET inhibitor.

Inhibition of BET proteins was shown to be an effective mode of intervention in rodent models of human NUT midline carcinoma, multiple myeloma, Burkitt's lymphoma and acute myeloid leukemia by suppressing the expression of MYC gene (Nature. 2010 Dec. 23; 468(7327):1067-73; Cell. 2011 Sep. 16; 146(6):904-1; Proc Natl Acad Sci USA. 2011 Oct. 4; 108(40):16669-74), as well as MYCN gene (Cancer Discov. 2013 Mar. 3(3) 308-23). MYC and homologous genes are some of the most overexpressed genes in human cancers; however, there has not been a pharmaceutical compound that directly antagonizes the activity of proteins encoded by the MYC gene and homologous genes to date partly due to the lack of effective drug binding sites. Thus, there exists a need for a means of indirect suppression of the expression of the MYC and homologous genes by inhibiting bromodomains of BET proteins which provide an effective mode of treatment for various diseases, disorders or medical conditions, including various cancers.

### SUMMARY OF THE INVENTION

The present invention includes compounds which bind to bromodomain-containing proteins and subsequently modulate the binding of acetylated proteins to bromodomain-containing proteins. In one aspect, the invention provides compounds of Formulae I and II,

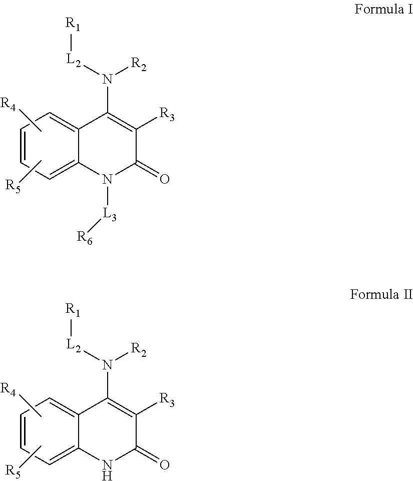

wherein each of R₁, R₂, R₃, R₄, R₅, R₆, L₁, L₂ and L₃ is as defined and described in embodiments herein, and pharmaceutically acceptable salts, solvates, polymorphs, isomers and prodrugs thereof.

### DETAILED DESCRIPTION OF THE INVENTION

According to one aspect of the invention, a compound having the structure of Formula I, and pharmaceutically acceptable salts, solvates, polymorphs, isomers or prodrugs thereof, is provided

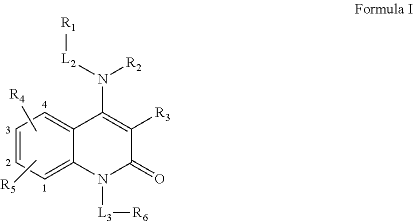

wherein:

L₂ is —N(R₇)— or —(NC(O)R₇)—;

R₁ is selected from the group consisting of alkyl, cycloalkyl, heterocycloalkyl, aryl and heteroaryl, wherein said heteroaryl or heterocycloalkyl include one or more nitrogen (N), oxygen (O) or sulfur (S) atoms; wherein said alkyl, cycloalkyl, heterocycloalkyl, aryl and heteroaryl are optionally substituted with one or more R₁₂;

Optionally, R₁ and R₇ may be taken together with the attached nitrogen to form a 5- to 7-membered heterocycloalkyl ring, optionally fused with an aryl group or unfused, wherein said heterocycloalkyl ring may contain an additional one or more N, O or S atoms; wherein said heterocycloalkyl ring may optionally be substituted at any position by one or more R₁₂;

R₂ is H, alkyl, —C(O)R₇, —CH₂C(O)OR₇, —OC(O)NR₇R₈, —C(O)NR₇R₈ or —C(O)OR₇, wherein said alkyl is optionally substituted with one or more R₁₂;

L₃ is a bond, —(CR₁₀R₁₁)ₙ—, —C(O)NR₁₀—, —S(O)₂NR₁₀—, —R₁₀C(O)NR₁₁—, or —OC(O)NR₁₀; and n is 0, 1, 2, or 3;

R₃ is hydrogen (H) or alkyl, wherein said alkyl is optionally substituted with one or more R₁₂;

R₄ and R₅ are independently selected for each occurrence from the group consisting of H, alkyl, halogen, CN, CF₃, NO₂, C(O)OR₇, OC(O)NR₇R₈, C(O)NR₇R₈, NR₇R₈, NR₇C(O)R₈, NR₇C(O)OR₈NR₇S(O)₂R₈, NR₇C(O)NR₈R₉ aryl, heteroaryl, cycloalkyl and heterocycloalkyl, wherein said heterocycloalkyl or heteroaryl include one or more N, O or S atoms; wherein said alkyl, aryl, heteroaryl, cycloalkyl and heterocycloalkyl are each optionally substituted with one or more R₁₂;

R₆ is selected from the group consisting of H, alkyl, aryl, heteroaryl, cycloalkyl and heterocycloalkyl, wherein said heterocycloalkyl or heteroaryl include one or more N, O or S atoms; wherein said alkyl, aryl, heteroaryl, cycloalkyl and heterocycloalkyl are each optionally substituted with one or more R₁₂;

R₇, R₈ and R₉ are independently selected for each occurrence from the group consisting of H, alkyl, heteroalkyl, aryl, arylalkyl, heteroaryl, heteroarylalkyl, cycloalkyl and heterocycloalkyl, wherein said alkyl, heteroalkyl, aryl, arylalkyl, heteroaryl, heteroarylalkyl, cycloalkyl and heterocycloalkyl are each optionally substituted with one or more R₁₂;

Optionally, R₇ and R₈, together with the included atoms, may form a 4-, 5-, 6- or 7-membered cyclic ring system, wherein said cyclic ring system is a mono or bicyclic ring optionally having an additional from one to four heteroatoms selected from N, O and S; wherein said cyclic ring system is optionally substituted with one or more of hydroxyl, sulfhydryl, alkoxy, thioalkoxy, alkyl or halogen;

Optionally, R₈ and R₉, together with the included atoms, may form a 4-, 5-, 6- or 7-membered cyclic ring system, wherein said cyclic ring system is a mono or bicyclic ring optionally having an additional from one to four heteroatoms selected from N, O and S; wherein said cyclic ring system is optionally substituted with one or more of hydroxyl, sulfhydryl, alkoxy, thioalkoxy, alkyl or halogen;

R₁₀ and R₁₁ are independently selected for each occurrence from H or alkyl, wherein said alkyl may optionally be substituted with one or more R₁₂;

and

R₁₂ is independently selected for each occurrence from the group consisting of lower (C₁-C₆) alkyl, lower alkenyl, lower alkynyl, aryl, heteroaryl, alicyclic, heterocyclic, arylalkyl, heteroarylalkyl, alkoxy, aryloxy, amino, alkylamino, dialkylamino, diarylalkylamino, alkylthio, arylthio, heteroarylthio, oxo, oxa, —C(O)—, —C(O)OR, —C(O)NH₂, —CO₂H, acyloxy, H, halo, —CN, —NO₂, —N₃, —SH, —OH, —C(O)CH₃, perhaloalkyl, perhaloalkoxy, perhaloacyl, guanidinyl, pyridinyl, thiophene, furanyl, indolyl, indazolyl, phosphonate, phosphonic acid, phosphate, phosphoramide, sulfonate, sulfone, sulfate, sulphonamide, carbamate, urea, thiourea, thioamide and thioalkyl.

According to one embodiment, R₁ is aryl or heteroaryl, wherein said aryl or heteroaryl is optionally substituted with one or more R₁₂.

According to one embodiment, R₂ is hydrogen, alkyl, —C(O)R₇, or —CH₂C(O)OR₇ wherein said alkyl may optionally be substituted with one or more R₁₂.

According to one embodiment, R₄ and R₅ are independently selected from H, aryl, heteroaryl or heterocycloalkyl, wherein said heterocycloalkyl includes one or more N or O atoms; wherein said aryl, heteroaryl or heterocycloalkyl may optionally be substituted with one or more R₁₂.

According to one embodiment, L₂ is —NR₇.

According to one embodiment, L₂ is —(NC(O)R₇)—.

According to one embodiment, L₃ is a bond, H, or —(CR₁₀R₁₁)ₙ—, where R₁₀, R₁₁ and n are as previously defined.

According to one embodiment, the invention provides compounds of Formula I wherein L₂ is NR₇ (compounds of Formula III), selected from but not limited by the following Table (Table 1):

TABLE 1
Formula III

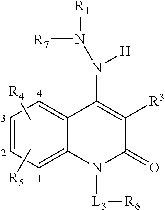

| Example | | | | Position, | Position, | | |
|---|---|---|---|---|---|---|---|
| # | R₁ | R₇ | R₃ | R₄ | R₅ | L₃ | R₆ |
| 1 | 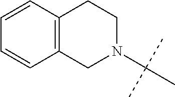 | | H | 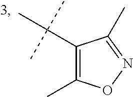 | H | bond | H |
| 2 | 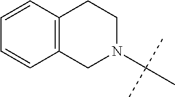 | | CH₃ | 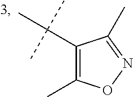 | H | bond | H |
| 3 | 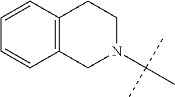 | | H | H | 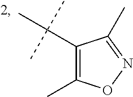 | bond | H |
| 4 | 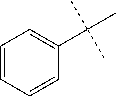 | H | H | 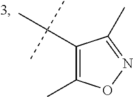 | H | bond | H |
| 5 | 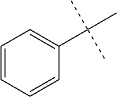 | CH₃ | H | 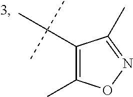 | H | bond | H |
| 6 | 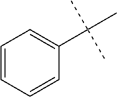 | CH₂COOH | H | 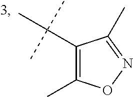 | H | bond | H |
| 7 | 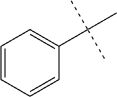 | COCH₃ | H | 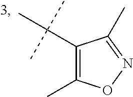 | H | bond | H |
| 8 | 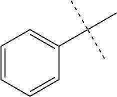 | H | H | 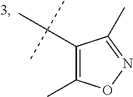 | H | —CH2— | H |
| 9 | 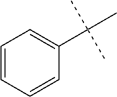 | CH₃ | H | 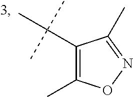 | H | —CH2— | H |
| 10 | 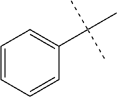 | CH₂COOH | H | 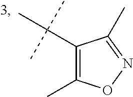 | H | —CH2— | H |
| 11 | 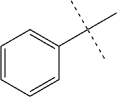 | COCH₃ | H | 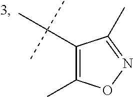 | H | —CH2— | H |
| 12 | 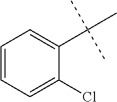 | H | H | 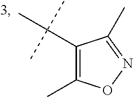 | H | bond | H |
| 13 | 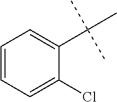 | CH₃ | H | 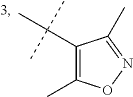 | H | bond | H |
| 14 | 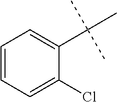 | CH₂COOH | H | 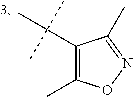 | H | bond | H |
| 15 | 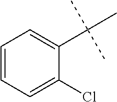 | COCH₃ | H | 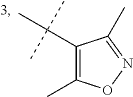 | H | bond | H |
| 16 | 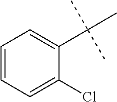 | H | H | 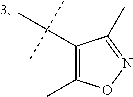 | H | —CH2— | H |
| 17 | 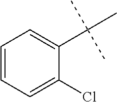 | CH₃ | H | 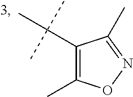 | H | —CH2— | H |
| 18 | 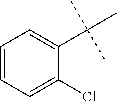 | CH₂COOH | H | 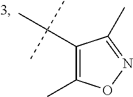 | H | —CH2— | H |
| 19 | 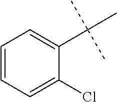 | COCH₃ | H | 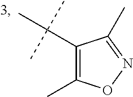 | H | —CH2— | H |
| 20 | 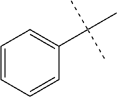 | H | H | 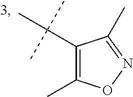 | H | bond | H |
| 21 | 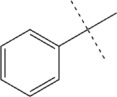 | CH₃ | CH₃ | 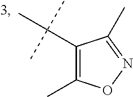 | H | bond | H |
| 22 | 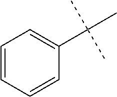 | CH₂COOH | CH₃ | 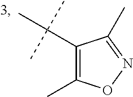 | H | bond | H |
| 23 | 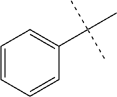 | COCH₃ | CH₃ | 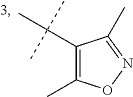 | H | bond | H |
| 24 | 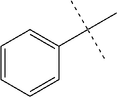 | H | CH₃ | 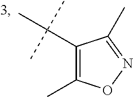 | H | —CH2— | H |
| 25 | 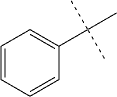 | CH₃ | CH₃ | 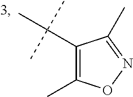 | H | —CH2— | H |
| 26 | 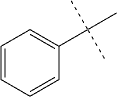 | CH₂COOH | CH₃ | 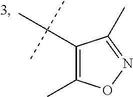 | H | —CH2— | H |
| 27 | 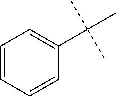 | COCH₃ | CH₃ | 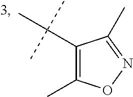 | H | —CH2— | H |
| 28 | 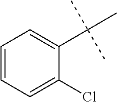 | H | CH₃ | 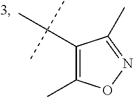 | H | bond | H |
| 29 | 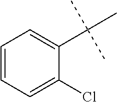 | CH₃ | CH₃ | 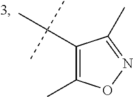 | H | bond | H |
| 30 | 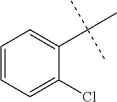 | CH₂COOH | CH₃ | 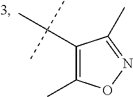 | H | bond | H |
| 31 | 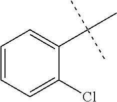 | COCH₃ | CH₃ | 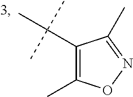 | H | bond | H |
| 32 | 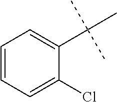 | H | CH₃ | 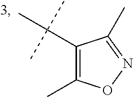 | H | —CH2— | H |
| 33 | 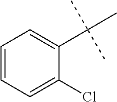 | CH₃ | CH₃ | 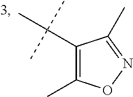 | H | —CH2— | H |
| 34 | 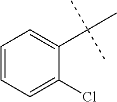 | CH₂COOH | CH₃ | 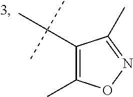 | H | —CH2— | H |
| 35 | 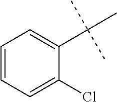 | COCH₃ | CH₃ | 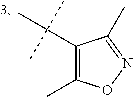 | H | —CH2— | H |
| 36 | 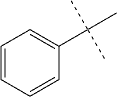 | H | H | 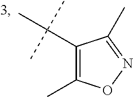 | 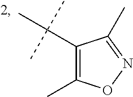 | bond | H |
| 37 | 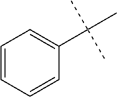 | CH₃ | H | 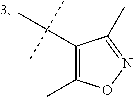 | 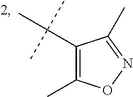 | bond | H |
| 38 | 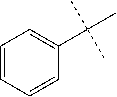 | CH₂COOH | H | 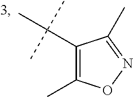 | 2, Ph | bond | H |
| 39 | 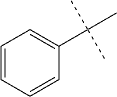 | COCH₃ | H | 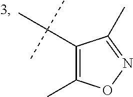 | 2, Ph | bond | H |
| 40 | 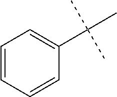 | H | H | 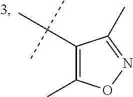 | 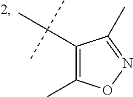 | —CH2— | H |
| 41 | 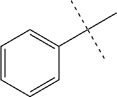 | CH₃ | H | 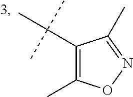 | 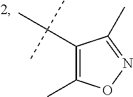 | —CH2— | H |
| 42 | 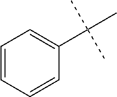 | CH₂COOH | H | 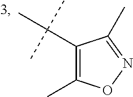 | 2, Ph | —CH2— | H |
| 43 | 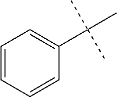 | COCH₃ | H | 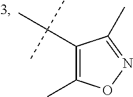 | 2, Ph | —CH2— | H |
| 44 | 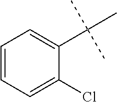 | H | H | 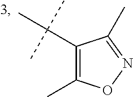 | 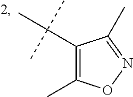 | bond | H |
| 45 | 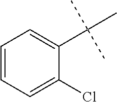 | CH₃ | H | 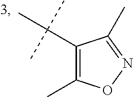 | 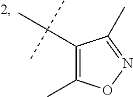 | bond | H |
| 46 | 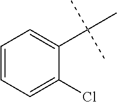 | CH₂COOH | H | 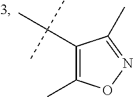 | 2, Ph | bond | H |
| 47 | 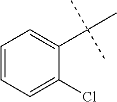 | COCH₃ | H | 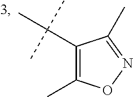 | 2, Ph | bond | H |
| 48 | 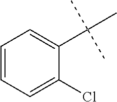 | H | H | 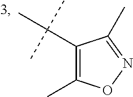 | 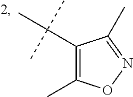 | —CH2— | H |
| 49 | 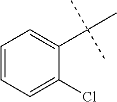 | CH₃ | H | 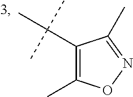 | 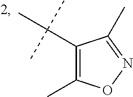 | —CH2— | H |
| 50 | 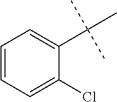 | CH₂COOH | H | 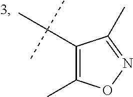 | 2, Ph | —CH2— | H |
| 51 | 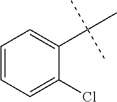 | COCH₃ | H | 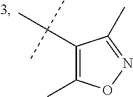 | 2, Ph | —CH2— | H |
| 52 | 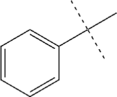 | H | H | 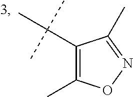 | 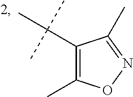 | bond | H |
| 53 | 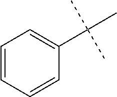 | CH₃ | CH₃ | 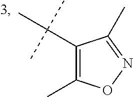 | 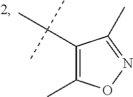 | bond | H |
| 54 | 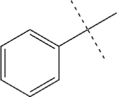 | CH₂COOH | CH₃ | 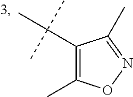 | 2, Ph | bond | H |
| 55 | 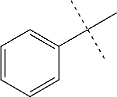 | COCH₃ | CH₃ | 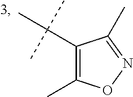 | 2, Ph | bond | H |
| 56 | 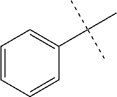 | H | CH₃ | 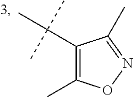 | 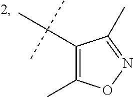 | —CH2— | H |
| 57 | 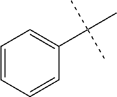 | CH₃ | CH₃ | 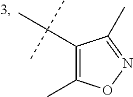 | 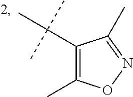 | —CH2— | H |
| 58 | 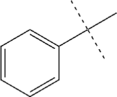 | CH₂COOH | CH₃ | 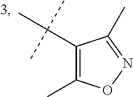 | 2, Ph | —CH2— | H |
| 59 | 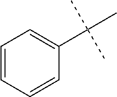 | COCH₃ | CH₃ | 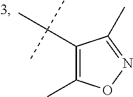 | 2, Ph | —CH2— | H |
| 60 | 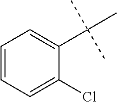 | H | CH₃ | 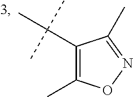 | 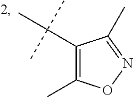 | bond | H |
| 61 | 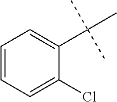 | CH₃ | CH₃ | 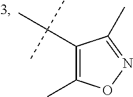 | 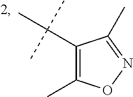 | bond | H |
| 62 | 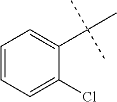 | CH₂COOH | CH₃ | 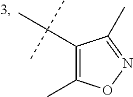 | 2, Ph | bond | H |
| 63 | 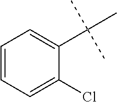 | COCH₃ | CH₃ | 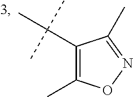 | 2, Ph | bond | H |
| 64 | 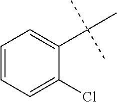 | H | CH₃ | 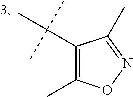 | 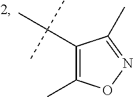 | —CH2— | H |
| 65 | 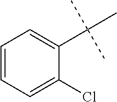 | CH₃ | CH₃ | 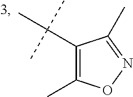 | 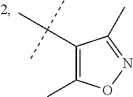 | —CH2— | H |
| 66 | 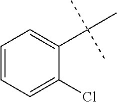 | CH₂COOH | CH₃ | 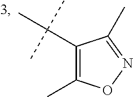 | 2, Ph | —CH2— | H |
| 67 | 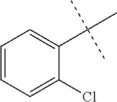 | COCH₃ | CH₃ | 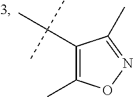 | 2, Ph | —CH2— | H |
| 68 | 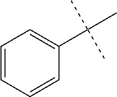 | H | H | 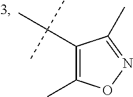 | H | —CH2— | Ph |
| 69 | 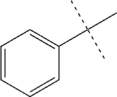 | CH₃ | H | 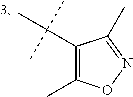 | H | —CH2— | Ph |
| 70 | 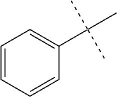 | CH₂COOH | H | 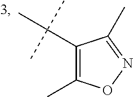 | H | —CH2— | Ph |
| 71 | 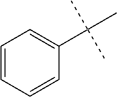 | COCH₃ | H | 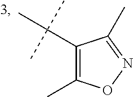 | H | —CH2— | Ph |
| 72 | 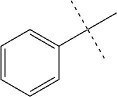 | H | H | 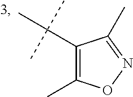 | H | —CH2— | Ph |
| 73 | 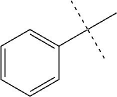 | CH₃ | H | 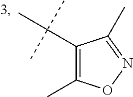 | H | —CH2— | Ph |
| 74 | 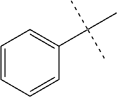 | CH₂COOH | H | 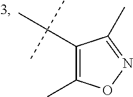 | H | —CH2— | Ph |
| 75 | 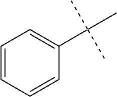 | COCH₃ | H | 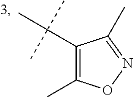 | H | —CH2— | Ph |
| 76 | 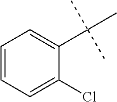 | H | H | 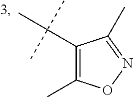 | H | —CH2— | Ph |
| 77 | 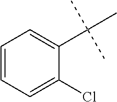 | CH₃ | H | 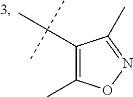 | H | —CH2— | Ph |
| 78 | 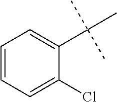 | CH₂COOH | H | 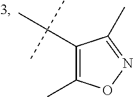 | H | —CH2— | Ph |
| 79 | 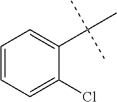 | COCH₃ | H | 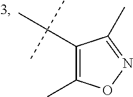 | H | —CH2— | Ph |
| 80 | 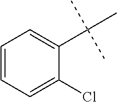 | H | H | 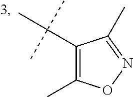 | H | —CH2— | Ph |
| 81 | 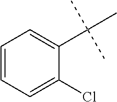 | CH₃ | H | 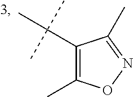 | H | —CH2— | Ph |
| 82 | 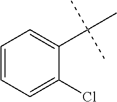 | CH₂COOH | H | 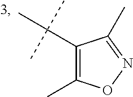 | H | —CH2— | Ph |
| 83 | 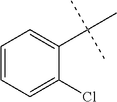 | COCH₃ | H | 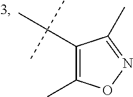 | H | —CH2— | Ph |
| 84 | 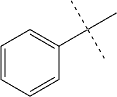 | H | H | 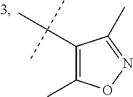 | H | —CH2— | Ph |
| 85 | 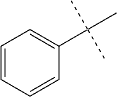 | CH₃ | CH₃ | 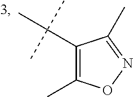 | H | —CH2— | Ph |
| 86 | 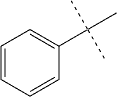 | CH₂COOH | CH3 | 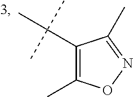 | H | —CH2— | Ph |
| 87 | 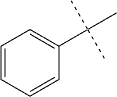 | COCH₃ | CH₃ | 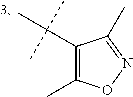 | H | —CH2— | Ph |
| 88 | 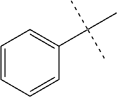 | H | CH₃ | 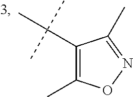 | H | —CH2— | Ph |
| 89 | 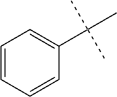 | CH₃ | CH₃ | 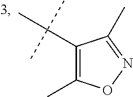 | H | —CH2— | Ph |
| 90 | 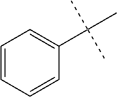 | CH₂COOH | CH₃ | 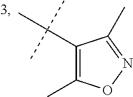 | H | —CH2— | Ph |
| 91 | 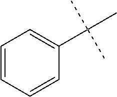 | COCH₃ | CH₃ | 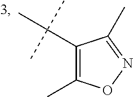 | H | —CH2— | Ph |
| 92 | 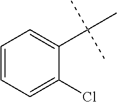 | H | CH₃ | 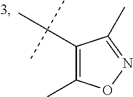 | H | —CH2— | Ph |
| 93 | 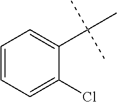 | CH₃ | CH₃ | 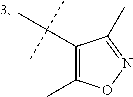 | H | —CH2— | Ph |
| 94 | 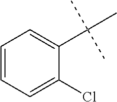 | CH₂COOH | CH₃ | 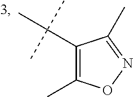 | H | —CH2— | Ph |
| 95 | 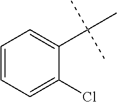 | COCH₃ | CH₃ | 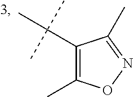 | H | —CH2— | Ph |
| 96 | 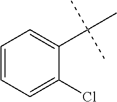 | H | CH₃ | 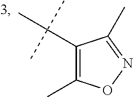 | H | —CH2— | Ph |
| 97 | 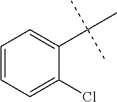 | CH₃ | CH₃ | 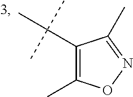 | H | —CH2— | Ph |
| 98 | 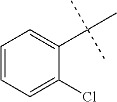 | CH₂COOH | CH₃ | 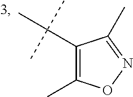 | H | —CH2— | Ph |
| 99 | 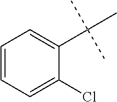 | COCH₃ | CH₃ | 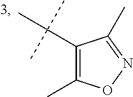 | H | —CH2— | Ph |
| 100 | 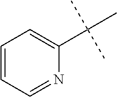 | H | H | 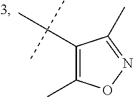 | H | —CH2— | Ph |
| 101 | 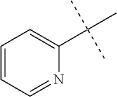 | CH₃ | H | 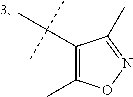 | H | —CH2— | Ph |
| 102 | 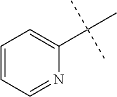 | CH₂COOH | H | 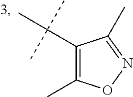 | H | —CH2— | H |
| 103 | 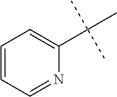 | COCH₃ | H | 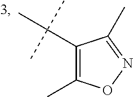 | H | —CH2— | H |
| 104 | 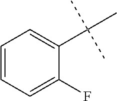 | H | H | 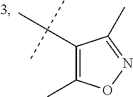 | H | bond | H |
| 105 | 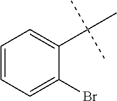 | H | H | 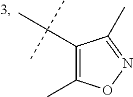 | H | bond | H |
| 106 | 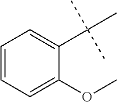 | H | H | 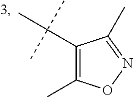 | H | bond | H |
| 107 | 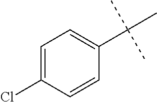 | H | H | 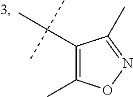 | H | bond | H |

In another aspect of the invention, a compound having a structure of Formula II,

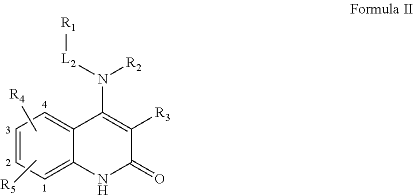

including pharmaceutically acceptable salts, solvates, polymorphs, isomers and prodrugs thereof, is provided wherein:

L₁ is a bond or is —(CR₁₀R₁₁)ₘ—; and m is 0, 1, 2, or 3;

R₁ is selected from the group consisting of alkyl, cycloalkyl, heterocycloalkyl, aryl and heteroaryl, wherein each of said heterocycloalkyl and heteroaryl include one or more N, O or S atoms; wherein each of said alkyl, cycloalkyl, heterocycloalkyl, aryl and heteroaryl are optionally substituted with one or more R₁₂;

R₂ is H, alkyl, —C(O)R₇, —CH₂C(O)OR₇, —OC(O)NR₇R₈, —C(O)NR₇R₈ or —C(O)OR₇, wherein said alkyl is optionally substituted with one or more R₁₂;

R₃ is H or alkyl, wherein said alkyl is optionally substituted with one or more R₁₂;

R₄ and R₅ are independently selected for each occurrence from the group consisting of H, alkyl, halogen, CN, CF₃, NO₂, C(O)OR₇, OC(O)NR₇R₈, C(O)NR₇R₈, NR₇R₈, NR₇C(O)R₈, NR₇C(O)OR₈NR₇S(O)₂R₈, NR₇C(O)NR₈R₉ aryl, heteroaryl, cycloalkyl and heterocycloalkyl, wherein said heterocycloalkyl and heteroaryl include one or more N, O or S atoms; wherein each of said alkyl, aryl, heteroaryl, cycloalkyl or heterocycloalkyl may optionally be substituted with one or more R₁₂;

R₇, R₈ and R₉ are independently selected for each occurrence from the group consisting of H, alkyl, heteroalkyl, aryl, arylalkyl, heteroaryl, heteroarylalkyl, cycloalkyl and heterocycloalkyl, wherein each of said alkyl, heteroalkyl, aryl, arylalkyl, heteroaryl, heteroarylalkyl, cycloalkyl and heterocycloalkyl groups may optionally be substituted with one or more R₁₂;

Optionally, R₇ and R₈, together with the included atoms, may form a 4-, 5-, 6- or 7-membered cyclic ring system, wherein said cyclic ring system is a mono or bicyclic ring optionally having an additional from one to four heteroatoms selected from N, O and S; wherein said cyclic ring system is optionally substituted with one or more R₁₂;

Optionally, R₈ and R₉, together with the included atoms, may form a 4-, 5-, 6- or 7-membered cyclic ring system, wherein said cyclic ring system is a mono or bicyclic ring optionally having an additional from one to four heteroatoms selected from N, O and S; wherein said cyclic ring system is optionally substituted with one or more R₁₂;

R₁₀ and R₁₁ are independently chosen for each occurrence from H or alkyl, wherein said alkyl may optionally be substituted with one or more R₁₂;

and

R₁₂ is independently selected for each occurrence from the group consisting of lower (C₁-C₆) alkyl, lower alkenyl, lower alkynyl, aryl, heteroaryl, alicyclic, heterocyclic, arylalkyl, heteroarylalkyl, alkoxy, aryloxy, amino, alkylamino, dialkylamino, diarylalkylamino, alkylthio, arylthio, heteroarylthio, oxo, oxa, —C(O)—, —C(O)OR, —C(O)NH₂, CO₂H, acyloxy, H, halo, —CN, —NO₂, —N₃, —SH, —OH, —C(O)CH₃, perhaloalkyl, perhaloalkoxy, perhaloacyl, guanidinyl, pyridinyl, thiophene, furanyl, indolyl, indazolyl, phosphonate, phosphonic acid, phosphate, phosphoramide, sulfonate, sulfone, sulfate, sulphonamide, carbamate, urea, thiourea, thioamide and thioalkyl.

According to one embodiment, R₁ is aryl, heteroaryl, cycloalkyl, or heterocycloalkyl, wherein said heteroaryl or heterocycloalkyl groups include one or more N, O or S atoms; wherein said aryl, heteroaryl, cycloalkyl, or heterocycloalkyl may each be optionally substituted with one or more of alkoxy, halogen, or aryl.

According to one embodiment, R₂ is H or alkyl.

According to one embodiment, R₃ is H.

According to one embodiment, R₄ and R₅ are independently selected for each occurrence from the group consisting of alkyl, aryl, heteroaryl, —C(O)NH₂ and —C(O)NH(CH₂)ₖOH, wherein k is 2 or 3; wherein said alkyl, aryl or heteroaryl is optionally substituted with one or more R₁₂.

According to one embodiment, compounds of Formula II, selected from but not limited by the following Table (Table 2), are provided:

TABLE 2
Formula II

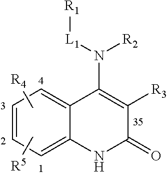

| Example | | | | | Position, | Position, |
|---|---|---|---|---|---|---|
| # | R₁ | L₁ | R₂ | R₃ | R₄ | R₅ |
| 108 | 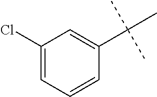 | —CH2— | H | H | 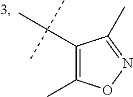 | H |
| 109 | 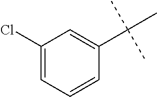 | —CH2— | CH₃ | H | 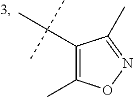 | H |
| 110 | 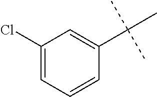 | (R); —CH(CH₃)— | H | H | 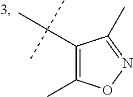 | H |
| 11 | 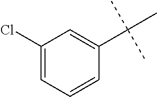 | (S); —CH(CH₃)— | CH₃ | H | 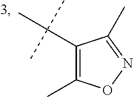 | H |
| 112 | 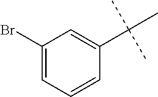 | —CH2— | H | H | 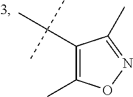 | H |
| 113 | 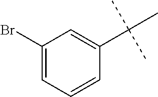 | —CH2— | CH₃ | H | 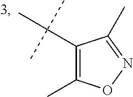 | H |
| 114 | 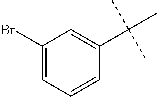 | (R); —CH(CH₃)— | H | H | 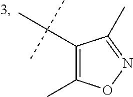 | H |
| 115 | 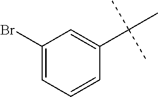 | (S); —CH(CH₃)— | CH₃ | H | 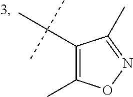 | H |
| 116 | 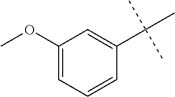 | —CH2— | H | H | 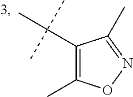 | H |
| 117 | 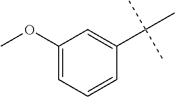 | —CH2— | CH₃ | H | 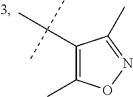 | H |
| 118 | 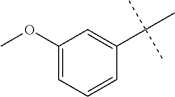 | (R); —CH(CH₃)— | H | H | 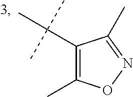 | H |
| 119 | 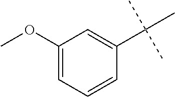 | (S); —CH(CH₃)— | CH₃ | H | 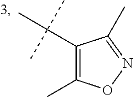 | H |
| 120 | 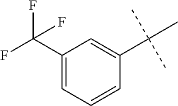 | —CH2— | H | H | 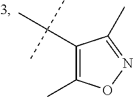 | H |
| 121 | 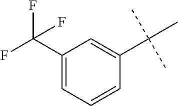 | —CH2— | CH₃ | H | 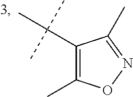 | H |
| 122 | 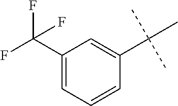 | (R); —CH(CH₃)— | H | H | 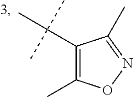 | H |
| 123 | 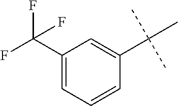 | (S); —CH(CH₃)— | CH₃ | H | 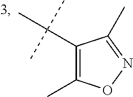 | H |
| 124 | 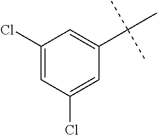 | —CH2— | H | H | 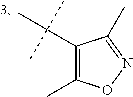 | H |
| 125 | 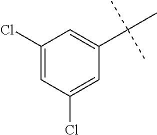 | —CH2— | CH₃ | H | 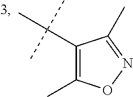 | H |
| 126 | 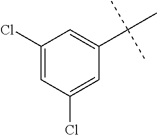 | (R); —CH(CH₃)— | H | H | 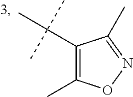 | H |
| 127 | 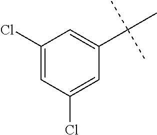 | (S); —CH(CH₃)— | CH₃ | H | 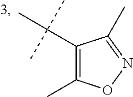 | H |
| 128 | 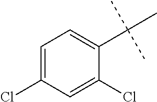 | —CH2— | H | H | 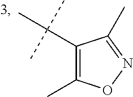 | H |
| 129 | 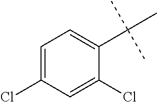 | —CH2— | CH₃ | H | 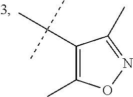 | H |
| 130 | 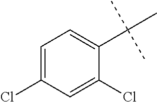 | (R); —CH(CH₃)— | H | H | 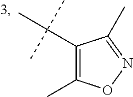 | H |
| 131 | 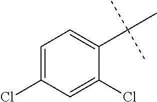 | (S); —CH(CH₃)— | CH₃ | H | 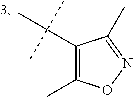 | H |
| 132 | 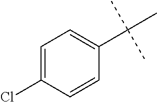 | —CH2— | H | H | 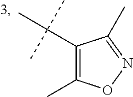 | H |
| 133 | 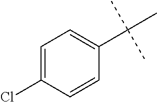 | —CH2— | CH₃ | H | 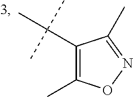 | H |
| 134 | 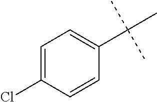 | (R); —CH(CH₃)— | H | H | 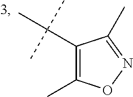 | H |
| 135 | 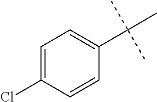 | (S); —CH(CH₃)— | CH₃ | H | 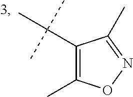 | H |
| 136 | 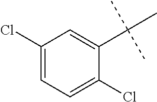 | —CH2— | H | H | 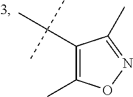 | H |
| 137 | 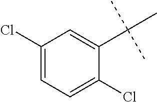 | —CH2— | CH₃ | H | 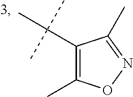 | H |
| 138 | 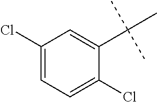 | (R); —CH(CH₃)— | H | H | 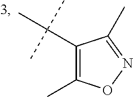 | H |
| 139 | 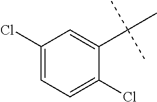 | (S); —CH(CH₃)— | CH₃ | H | 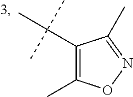 | H |
| 140 | 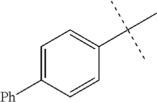 | —CH2— | H | H | 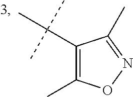 | H |
| 141 | 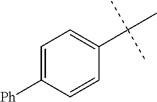 | —CH2— | CH₃ | H | 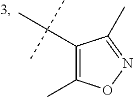 | H |
| 142 | 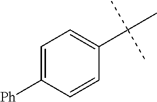 | (R); —CH(CH₃)— | H | H | 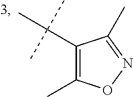 | H |
| 143 | 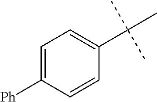 | (S); —CH(CH₃)— | CH₃ | H | 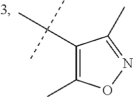 | 2, Ph |
| 144 | 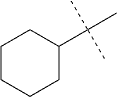 | —CH2— | H | H | 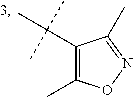 | H |
| 145 | 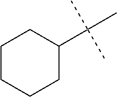 | —CH2— | CH₃ | H | 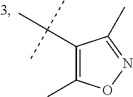 | H |
| 146 | 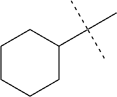 | (R); —CH(CH₃)— | H | H | 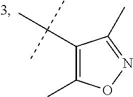 | H |
| 147 | 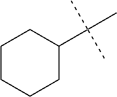 | (S); —CH(CH₃)— | CH₃ | H | 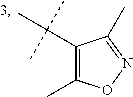 | H |
| 148 | 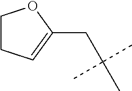 | —CH2— | H | H | 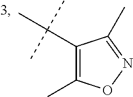 | H |
| 149 | 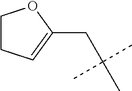 | —CH2— | CH₃ | H | 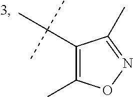 | H |
| 150 | 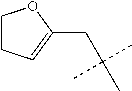 | (R); —CH(CH₃)— | H | H | 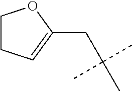 | 2, CH₃ |
| 151 | 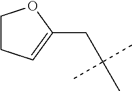 | (S); —CH(CH₃)— | CH₃ | H | 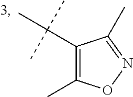 | 2, CH₃ |
| 152 | 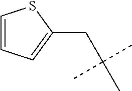 | —CH2— | H | H | 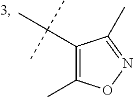 | H |
| 153 | 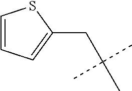 | —CH2— | CH₃ | H | 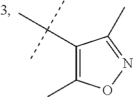 | H |
| 154 | 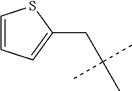 | (R); —CH(CH₃)— | H | H | 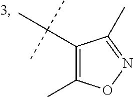 | H |
| 155 | 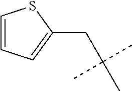 | (S); —CH(CH₃)— | CH₃ | H | 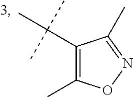 | H |
| 156 | 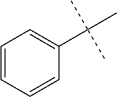 | (R); —C(H)(CH₂CH₃)— | H | H | 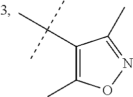 | H |
| 157 | 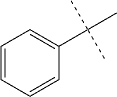 | (S); —C(H)(CH₂CH₃)— | CH₃ | H | 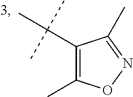 | H |
| 158 | 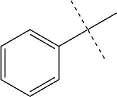 | (R); —C(H)(CH₂CH₃)— | H | H | 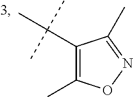 | H |
| 159 | 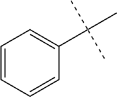 | (S); —C(H)(CH₂CH₃)— | CH₃ | H | 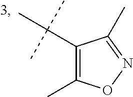 | H |
| 160 | 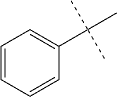 | —CH2— | H | H | 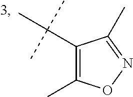 | 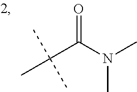 |
| 161 | 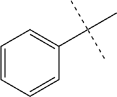 | —CH2— | H | H | 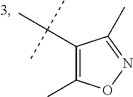 | 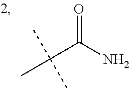 |
| 162 | 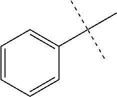 | —CH2— | H | H | 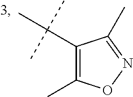 | 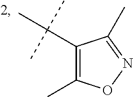 |
| 163 | 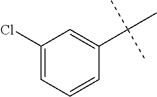 | —CH2— | H | H | 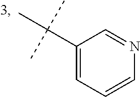 | H |
| 164 | 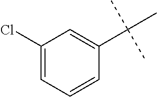 | —CH2— | H | H | 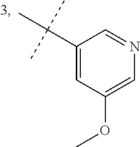 | H |
| 165 | 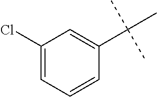 | —CH2— | H | H | 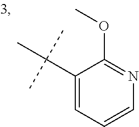 | H |
| 166 | 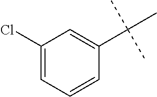 | —CH2— | H | H | 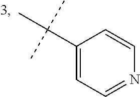 | H |
| 167 | 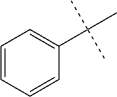 | —CH2— | H | H | 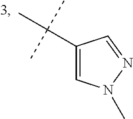 | H |
| 168 | 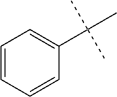 | —CH2— | H | H | 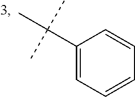 | H |
| 169 | 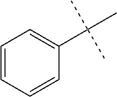 | —CH2— | H | H | 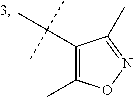 | 1, F |
| 170 | 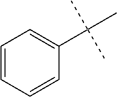 | —CH2— | H | H | 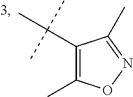 | H |
| 171 | 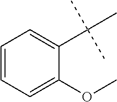 | —CH2— | H | H | 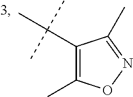 | H |
| 172 | 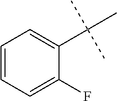 | —CH2— | H | H | 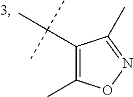 | H |
| 173 | 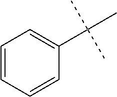 | (R); —CH(CH₃)— | H | H | 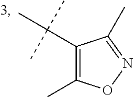 | H |
| 174 | 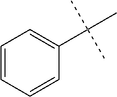 | (S); —CH(CH₃)— | CH₃ | H | 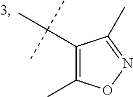 | H |
| 175 | 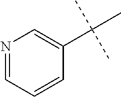 | —CH2— | H | H | 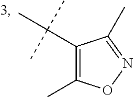 | H |
| 176 | 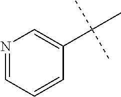 | —CH2— | CH₃ | H | 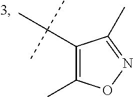 | H |
| 177 | 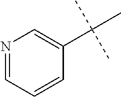 | (R); —CH(CH₃)— | H | H | 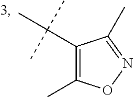 | H |
| 178 | 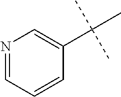 | (S); —CH(CH₃)— | CH₃ | H | 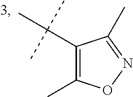 | H |
| 179 | 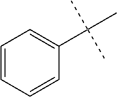 | —CH2— | H | H | 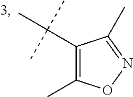 | 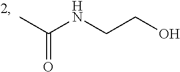 |

Certain of the compounds described herein contain one or more chiral centers, or may otherwise be capable of existing as multiple stereoisomers. The scope of the present invention includes mixtures of stereoisomers as well as purified enantiomers or enantiomerically and/or diastereomerically enriched mixtures. Also included within the scope of the present invention are the individual stereoisomers of the compounds represented by Formulae I, II and III, as well as any wholly or partially equilibrated mixtures thereof. The present invention also includes the individual stereoisomers of the compounds represented by the formulas above as mixtures with isomers thereof in which one or more chiral centers are inverted.

In one embodiment, compounds of the present invention are provided as pharmaceutically acceptable salts which include non-toxic salts of the compounds set forth herein. Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt.

The salts provided may be in some cases hydrates or solvates. The present invention includes a salt or solvate of the compounds herein described, including combinations thereof such as a solvate of a salt. The compounds of the present invention may exist in solvated, for example hydrated or ethanol complexed, as well as un-solvated forms, and the present invention encompasses all such forms. The salts of the present invention can be pharmaceutically acceptable salts.

The compounds or their pharmaceutically acceptable salts as provided herein may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Although it is possible to administer the compounds of the present invention in the form of a bulk active chemical, it is preferred to administer the compound in the form of a pharmaceutical composition or formulation. Thus, pharmaceutical compositions are provided that include one or more compounds of Formulae I, II or III and/or pharmaceutically acceptable salts thereof and one or more pharmaceutically acceptable carriers, diluents, or excipients.

Another embodiment of the invention provides a process for the preparation of a pharmaceutical composition including admixing one or more compounds of Formulae I, II, or III and/or pharmaceutically acceptable salts thereof with one or more pharmaceutically acceptable carriers, diluents or excipients.

According to one embodiment, compounds which bind to and otherwise modulate acetylated protein binding to bromodomain-containing proteins are provided. Such compounds include at least one compound selected from Formula I, II or III as provided herein. Exemplary compounds include, but are not limited to, those compounds set forth previously in Tables 1 and 2.

According to one embodimentt, compounds for use in the treatment or prevention of a disease or condition mediated by inhibiting bromodomain-containing proteins from binding acetylated proteins are provided.

According to another embodiment, compounds for use in the treatment or prevention of a disease or condition mediated by inhibiting acetylated proteins from binding bromodomain-containing proteins are provided.

According to one embodiment, a method for the treatment or prevention of a disease is provided that includes the step of administering a compound as provided herein to inhibit the activity of bromodomain-containing proteins.

According to another embodiment, a method for the treatment or prevention of a disease is provided that includes the step of administering a compound as provided herein to inhibit the activity of bromodomain-containing proteins by inhibiting binding to acetylated proteins.

According to another embodiment, the use of a compound or salt thereof, for the preparation of a pharmaceutical composition for the treatment or prevention of a disease or condition mediated by inhibiting bromodomain-containing proteins by inhibiting binding to acetylated proteins is provided. According to one embodiment, the acetylated protein is an acetylated histone.

According to one embodiment, the acetylated protein is an acetylated histone involved in the regulation or dysregulation of gene expression.

The compounds of the present invention, their pharmaceutically acceptable salts and their pharmaceutical compositions can be used for treating or preventing a wide variety of conditions or disorders.

According to one embodiment, the disease or condition subject to prevention or treatment includes human NUT midline carcinoma, multiple myeloma, Burkitt's lymphoma, myeloid leukemia, NPM1c mutant leukemia, T-cell lymphoblastic leukemia, hepatocellular carcinoma, glioblastoma, neuroblastoma, sarcoma, breast cancer, colorectal cancer, lung cancer, pancreatic cancer, neuroendocrine tumors, Merkel cell carcinoma, prostate cancer, osteoarthritis, rheumatoid arthritis, Alzheimer's disease, or HIV infection.

According to another embodiment, a method for the treatment or prevention of a disease or condition mediated by bromodomain-containing proteins is provided and includes the step of administering a compound as provided herein. Any of the methods or uses provided herein may include administering to a subject a therapeutically effective amount of a compound as provided herein, including a salt or polymorph thereof, or a pharmaceutical composition that includes such compounds.

The manner in which the compounds or their pharmaceutical composition set forth herein may be administered can vary. According to one embodiment, the compounds can be administered orally. Preferred pharmaceutical compositions may be formulated for oral administration in the form of tablets, capsules, caplets, syrups, solutions, and suspensions. Such oral formulations can be provided in modified release dosage forms such as time-release tablet and capsule formulations. Pharmaceutical compositions can also be administered via injection, namely, intravenously, intramuscularly, subcutaneously, intraperitoneally, intra-arterial, intrathecally, and intracerebroventricularly. Intravenous administration is a preferred method of injection. Suitable carriers for injection are well known to those of skill in the art and include 5% dextrose solutions, saline, and phosphate buffered saline.

Pharmaceutical compositions may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation); by powder injection; or by buccal, sublingual, or intranasal absorption. Pharmaceutical compositions may be formulated in unit dose form, or in multiple or subunit doses.

The administration of the pharmaceutical compositions described herein can be intermittent, or at a gradual, continuous, constant or controlled rate. The pharmaceutical compositions may be administered to a warm-blooded animal, for example, a mammal such as a human being. In addition, the time of day and the number of times per day that the pharmaceutical composition is administered can vary.

The compounds as provided herein may also be used for the preparation of a medicament for the treatment or prevention of a disease or condition characterized by bromodomain-containing proteins binding acetylated proteins and altering normal gene expression. Methods for treating, preventing, delaying the onset of, or slowing the progression of disorders mediated by acetylated proteins involved in the regulation or dysregulation of gene expression, in mammals in need of such treatment are also provided. The methods involve administering to a subject a therapeutically effective amount of a compound as provided herein, including a salt thereof, or a pharmaceutical composition that includes such compounds.

According to one embodiment, the methods for treating, preventing, delaying the onset of, or slowing the progression of disorders mediated by acetylated proteins involved in the regulation or dysregulation of gene expression, in mammals in need of such treatment include the administration of at least one compound as provided herein including, but not limited to, the compounds provided according to Formulae I, II and III.

The compounds alone or in a pharmaceutical composition as provided herein may be used in the treatment of a variety of disorders and conditions and, as such, may be used in combination with a variety of other suitable therapeutic agents useful in the treatment or prophylaxis of those disorders or conditions. Thus, one embodiment of the present invention includes the administration of the compound of the present invention in combination with other therapeutic compounds. Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other treatment agents may be in combination by administration concomitantly in: (1) a unitary pharmaceutical composition including two or more compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another embodiment of the present invention includes combination therapy comprising administering to the subject a therapeutically or prophylactically effective amount of the compound of the present invention and one or more other therapy including chemotherapy, radiation therapy, gene therapy, or immunotherapy.

The compounds of the present invention can be used to mediate the prevention or treatment of various conditions or disorders mediated by inhibiting bromodomain-containing proteins from binding acetylated proteins. The compounds and their pharmaceutical compositions are particularly useful in the treatment or prevention of various types of cancer, inflammation, obesity, metabolic, cardiovascular, neurodegenerative, psychiatric and infectious diseases.

According to one embodiment, the compounds and their pharmaceutical compositions are particularly useful in the treatment or prevention of systemic or tissue inflammation, inflammatory responses to infection or hypoxia, cellular activation and proliferation, lipid metabolism, fibrosis and viral infections.

According to one embodiment, the compounds and their pharmaceutical compositions are particularly useful in the treatment or prevention of a variety of chronic autoimmune and inflammatory conditions such as rheumatoid arthritis, osteoarthritis, acute gout, psoriasis, systemic lupus erythematosus, multiple sclerosis, inflammatory bowel disease (Crohn's disease and Ulcerative colitis), asthma, chronic obstructive airways disease, pneumonitis, myocarditis, pericarditis, myositis, eczema, dermatitis, alopecia, vitiligo, bullous skin diseases, nephritis, vasculitis, atherosclerosis, depression, retinitis, uveitis, scleritis, hepatitis, pancreatitis, primary biliary cirrhosis, sclerosing cholangitis, Addison's disease, hypophysitis, thyroiditis, type I diabetes and acute rejection of transplanted organs.

According to one embodiment, the compounds and their pharmaceutical compositions are particularly useful in the treatment or prevention of a wide variety of acute inflammatory conditions such as acute gout, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, vasculitis with organ involvement and acute rejection of transplanted organs.

According to one embodiment, the compounds and their pharmaceutical compositions are particularly useful in the treatment or prevention of diseases or conditions which involve inflammatory responses to infections with bacteria, viruses, fungi, parasites or their toxins, such as sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria and SIRS associated with viral infections such as influenza, herpes zoster, herpes simplex and coronavirus.

According to one embodiment, the compounds and their pharmaceutical compositions are particularly useful in the treatment or prevention of conditions associated with ischaemia-reperfusion injury such as myocardial infarction, cerebro-vascular ischaemia (stroke), acute coronary syndromes, renal reperfusion injury, organ transplantation, coronary artery bypass grafting, cardio-pulmonary bypass procedures, pulmonary, renal, hepatic, gastro-intestinal or peripheral limb embolism.

According to one embodiment, the compounds and their pharmaceutical compositions are particularly useful in the treatment or prevention of disorders of lipid metabolism via the regulation of APO-A1 such as hypercholesterolemia, atherosclerosis and Alzheimer's disease.

According to one embodiment, the compounds and their pharmaceutical compositions are particularly useful in the treatment or prevention of fibrotic conditions such as idiopathic pulmonary fibrosis, renal fibrosis, post-operative stricture, keloid formation, scleroderma and cardiac fibrosis.

According to one embodiment, the compounds and their pharmaceutical compositions are particularly useful in the treatment or prevention of viral infections such as herpes virus, human papilloma virus, adenovirus and poxyirus and other DNA viruses.

According to one embodiment, the compounds and their pharmaceutical compositions are particularly useful in the treatment or prevention of diseases associated with systemic inflammatory response syndrome including sepsis, burns, pancreatitis, major trauma, hemorrhage and ischaemia.

According to one embodiment, the compounds and their pharmaceutical compositions are particularly useful in the treatment or prevention of SIRS, the onset of shock, multi-organ dysfunction syndrome, which includes the onset of acute lung injury, ARDS, acute renal, hepatic, cardiac and gastro-intestinal injury and mortality.

According to one embodiment, the compounds and their pharmaceutical compositions are particularly useful in the treatment or prevention of sepsis, sepsis syndrome, septic shock and endotoxaemia, acute or chronic pancreatitis, herpes simplex infections and reactivations, cold sores, herpes zoster infections and reactivations, chickenpox, shingles, human papilloma virus, cervical neoplasia, adenovirus infections, including acute respiratory disease, poxyirus infections such as cowpox and smallpox and African swine fever virus and for the treatment of Human papilloma virus infections of skin or cervical epithelia.

According to one embodiment, the compounds and their pharmaceutical compositions are particularly useful in the treatment or prevention of various forms of cancer, leukemias and lymphomas including acute myeloid leukemia, NPM1c mutant leukemia, Burkitt's lymphoma, multiple myeloma, T-cell lymphoblastic leukemia and other hematological cancers that involve translocations of mixed-lineage leukemia gene (MLL); solid tumors such as hepatocellular carcinoma, glioblastoma, medulloblastoma, neuroblastoma, NUT midline carcinoma, sarcoma, breast, colorectal, lung, pancreatic, neuroendocrine tumors including those involving the pancreas and thymus (PanNETS and NETs), and Merkel cell carcinoma (MCC) and prostate cancer; osteoarthritis and rheumatoid arthritis;

Alzheimer's disease; and HIV infection.

It is contemplated and therefore within the scope of the present invention that any feature that is described above can be combined with any other feature that is described above. It is also contemplated and therefore within the scope of the present invention that negative provisos can be added to exclude any compound or remove any feature.

### Definitions

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well. Thus, for example, C₁₋₆ alkyl represents a straight or branched chain hydrocarbon containing one to six carbon atoms.

As used herein the term “alkyl” refers to a straight or branched chain hydrocarbon, which may be optionally substituted, with multiple degrees of substitution being allowed. The term “lower alkyl” refers to an alkyl that includes from one to six carbon atoms, Examples of “lower alkyl” as used herein include, but are not limited to, methyl, ethyl, propyl, isopropyl, isobutyl, n-butyl, tert-butyl, isopentyl, and n-pentyl.

As used herein, the term “alkene” or “alkenyl” group refers to an unsaturated hydrocarbon that includes one or more carbon-carbon double bonds. The term “lower alkene” refers to an alkene that includes from two to twenty carbon atoms, such as from two to ten carbon atoms. The term “substituted alkene” refers to an alkene that has one or more of its hydrogen atoms replaced by one or more substituent groups, such as halogen.

As used herein, the term “alkyne” or “alkynyl” group refers to an unsaturated hydrocarbon that includes one or more carbon-carbon triple bonds. The term “lower alkyne” refers to an alkyne that includes from two to twenty carbon atoms, such as from two to ten carbon atoms. The term “substituted alkyne” refers to an alkyne that has one or more of its hydrogen atoms replaced by one or more substituent groups, such as halogen.

As used herein, the term “cycloalkyl” refers to a fully saturated optionally substituted monocyclic, bicyclic, or bridged hydrocarbon ring, with multiple degrees of substitution being allowed. Preferably, the ring is three to twelve-membered, more preferably, from five- to six-membered. Exemplary “cycloalkyl” groups as used herein include, but are not limited to, cyclopropyl, cyclobutyl, cyclopentyl, cyclohexyl, and cycloheptyl.

As used herein, the term “alkoxy” refers to a group —OR^{a}, where R^{a} is “alkyl” as defined herein.

As used herein, the term “heterocycloalkyl” or “heterocycle” or “heterocyclyl” refers to an optionally substituted mono- or polycyclic ring system, optionally containing one or more degrees of unsaturation, and also containing one or more heteroatoms, which may be optionally substituted, with multiple degrees of substitution being allowed. Exemplary heteroatoms include nitrogen, oxygen, or sulfur atoms, including N-oxides, sulfur oxides, and dioxides. Preferably, the ring is three to twelve-membered, preferably five or six-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, tetrahydropyran, 1,4-dioxane, 1,3-dioxane, piperidine, pyrrolidine, morpholine, tetrahydrothiopyran, and tetrahydrothiophene.

As used herein, the term “aryl” refers to a single benzene ring or fused benzene ring system which may be optionally substituted, with multiple degrees of substitution being allowed. Examples of “aryl” groups as used include, but are not limited to, phenyl, benzyl, 2-naphthyl, 1-naphthyl, anthracene, and phenanthrene. Preferable aryl rings have five- to ten-members. The term “aryl” also includes a fused benzene ring system, namely where a cyclic hydrocarbon or heterocycle (e.g., a cyclohexane or dioxane ring) or heteroaryl (e.g., pyridine) is fused with an aromatic ring (aryl, such as a benzene ring).

As used herein, the term “heteroaryl” refers to a monocyclic five to seven membered aromatic ring, a fused bicyclic aromatic ring system comprising two of such aromatic rings, which may be optionally substituted, with multiple degrees of substitution being allowed, or to a fused bicyclic ring system namely where a cycloalkyl or heterocycle (e.g., a cyclohexane or dioxane ring) is fused with a heteroaryl ring. Preferably, heteroaryl rings contain five- to ten-members. These heteroaryl rings contain one or more nitrogen, sulfur, and/or oxygen atoms. In certain embodiments, the heteroaryl rings contain one to three nitrogen, one to three oxygen, or one or two sulfur atoms. N-oxides, sulfur oxides, and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinoxaline, benzofuran, benzoxazole, benzothiophene, indole, indazole, benzimidazole, imidazopyridine, pyrazolopyridine, and pyrazolopyrimidine.

As used herein the term “halogen” refers to fluorine, chlorine, bromine, or iodine.

As used herein the term “haloalkyl” refers to a substituted or unsubstituted alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein, the term “sulfhydryl” refers to refers to a —SH group.

As used herein, the term “thioalkoxy” refers to a group —SR^{a}, where R^{a} is “alkyl” as defined herein.

As used herein, the term “carboxyamido” refers to —NH—C(O)—W, wherein W is hydrogen or an unsubstituted or substituted alkyl, alkene, alkyne, cycloalkyl, aryl, or heterocycle group.

As used herein, the term “amine” is given its ordinary meaning and includes primary, secondary and tertiary amines.

As used herein, the term “amido” refers to a group of the formula —C(O)NR′R″, wherein R′ and R″ are substituted or unsubstituted alkyl, cycloalkyl or heterocycle, or R′ and R″ can form cycloalkyl or heterocycle. As used herein, the term “sulfamido” refers to the group —SO₂NR′R″.

As used herein, “optionally substituted”, groups may be substituted or unsubstituted. The substituent (or substitution) group may include, without limitation, one or more substituents independently selected from the following groups or designated subsets thereof: lower (C₁-C₆) alkyl, lower alkenyl, lower alkynyl, lower aryl, heteroaryl, alicyclic, heterocyclic, arylalkyl, heteroarylalkyl, lower alkoxy, lower aryloxy, amino, alkylamino, dialkylamino, diarylalkylamino, alkylthio, arylthio, heteroarylthio, oxo, oxa, carbonyl (—C(O)), carboxyesters (—C(O)OR), carboxamido (—C(O)NH₂), carboxy, acyloxy, —H, halo, —ON, —NO₂, —N₃, —SH, —OH, —C(O)CH₃, perhaloalkyl, perhaloalkoxy, perhaloacyl, guanidine, pyridinyl, thiophene, furanyl, indole, indazole, esters, amides, phosphonates, phosphonic acid, phosphates, phosphoramides, sulfonates, sulfones, sulfates, sulphonamides, carbamates, ureas, thioureas and thioamides, thioalkyls. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

As used herein, the term “pharmaceutically acceptable” refers to carrier(s), diluent(s), excipient(s) or salt forms of the compounds of the present invention that are compatible with the other ingredients of the formulation of the pharmaceutical composition.

As used herein, the term “pharmaceutical composition” refers to a compound of the present invention optionally admixed with one or more pharmaceutically acceptable carriers, diluents, or excipients. Pharmaceutical compositions preferably exhibit a degree of stability to environmental conditions so as to make them suitable for manufacturing and commercialization purposes.

As used herein, the terms “effective amount”, “therapeutic amount”, and “effective dose” refer to an amount of the compound of the present invention sufficient to elicit the desired pharmacological or therapeutic effects, thus resulting in an effective prevention or treatment of a disorder. Treatment of a disorder may be manifested by delaying or preventing the onset or progression of the disorder, as well as delaying or preventing the onset or progression of symptoms associated with the disorder. Treatment of a disorder may also be manifested by a decrease or elimination of symptoms, reversal of the progression of the disorder, as well as any other contribution to the well-being of the patient. The effective dose can vary, depending upon factors such as the condition of the patient, the severity of the symptoms of the disorder, and the manner in which the pharmaceutical composition is administered.

The term “prodrug” as used herein is intended to encompass a class of analogs of compounds of the present invention wherein a metabolically labile moiety is attached to said compound of the invention through an available NH, C(O)H, COOH, C(O)NH2, OH or SH functionality. The prodrug-forming moieties are removed by metabolic processes and release the active compounds having the free NH, C(O)H, COOH, C(O)NH2, OH or SH group in vivo. Prodrugs are useful for adjusting such pharmacokinetic properties of the compounds as solubility and/or hydrophobicity, absorption in the gastrointestinal tract, bioavailability, tissue penetration, and rate of clearance. Design and preparation of such prodrugs is known to those skilled in the art, and are described in: Various forms of prodrugs are well known in the art and are described in:
a) The Practice of Medicinal Chemistry, Camille G. Wermuth et al., Ch. 31 (Academic Press, 1996).
b) Design of Prodrugs, edited by H. Bundgaard, (Elsevier, 1985); 33.
c) A Textbook of Drug Design and Development, P. Krogsgaard-Larson and H. Bundgaard, eds. Ch. 5, pp. 113-191 (Harwood Academic Publishers, 1991); and
d) Hydrolysis in Drug and Prodrug Metabolism, Bernard Testa and Joachim M. Mayer, (Wiley-VCH, 2003).

Said references are incorporated herein by reference, particularly as to the description of prodrugs.

### Biological Assays

The following in vitro assays may be used to determine the activity of compounds of the present invention to interact with bromodomains and/or inhibit growth of MV4-11 acute myeloid leukemia cells. Representative data for a bromodomain assay are summarized in Table 3, thereby providing support for the utility of compounds of the present invention. Additionally, certain compounds possessed activity in a MV4-11 cell based assay as described below.

### Bromodomain Assays

Bromodomain assays were conducted to measure interactions between certain compounds and bromodomains. In order to measure compounds' direct activity against BRD4, dissociation constants (Ki values) of the bromodomain inhibitors of the present invention were obtained using a biophysical binding assay, BROMOscan℠ (DiscoveRx, San Diego, Calif.), a modified phage-display system originally developed for kinases (Nat Biotechnol. 2005 March; 23(3):329-36).

The assays were conducted according to BROMOscan protocol from Discover Rx. Briefly, BRD4 bromodomains expressed as a fusion capsid protein of T7 phage were bound to acetylated peptide ligand on a solid phase and competition bindings were performed. T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds were prepared as 1000× stocks in 100% DMSO and subsequently diluted 1:10 in monoethylene glycol (MEG) to create stocks at 100× the screening concentration (resulting stock solution is 10% DMSO/90% MEG). The compounds were then diluted directly into the assays such that the final concentration of DMSO and MEG were 0.1% and 0.9%, respectively. All reactions were performed in polystyrene 96-well plates in a final volume of 0.135 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 2 μM non-biotinylated affinity ligand) and incubated at room temperature with shaking for 30 minutes. The bromodomain concentration in the eluates was measured by qPCR. Data was collected at 11 compound concentrations ranging from 0 to 10 μM. Kd values were then derived from a standard dose-response curve generated with Hill equation (Hill Slope was set to −1) and a non-linear least square fit with the Levenberg-Marquardt algorithm.

Response=Background+Signal−Background/1+(Kd Hill Slope/Dose Hill Slope)

Data is reported in Table 3 as Relative Activity, wherein a Kd of less than 100 nM is denoted by (+++), a Kd of between 100 nM and 1 μM is denoted by (++), and a Kd of 1 μM to greater than 10 μM is denoted by (+).

TABLE 3

| Example # | BROMOscan Relative Activity |
|---|---|
| 1 | +++ |
| 5 | +++ |
| 6 | +++ |
| 7 | +++ |
| 60 | +++ |
| 108 | +++ |
| 112 | +++ |
| 116 | +++ |
| 120 | ++ |
| 124 | ++ |
| 128 | ++ |
| 132 | ++ |
| 140 | ++ |
| 144 | ++ |
| 148 | + |
| 152 | ++ |
| 160 | +++ |
| 161 | +++ |
| 162 | +++ |
| 163 | +++ |
| 164 | +++ |
| 165 | + |
| 166 | +++ |
| 167 | +++ |
| 170 | +++ |
| 171 | +++ |
| 172 | +++ |
| 173 | +++ |
| 175 | ++ |

### In Vitro Cell Viability

In order to further characterize the activity of bromodomain inhibitors of the present invention, a cell viability assay was performed using MV4-11 cells. Previously, the MV4-11 cell line, an acute myeloid leukemia cell line that contains a MLL-AF4 chromosomal translocation, was shown to be sensitive to a BET inhibitor (Nature. 2011 Oct. 2; 478 (7370):529-33). We independently confirmed a profound suppression of MYC gene expression following brief exposures to BET inhibitors (FIG. 1) in this cell line.

MV4-11 acute myeloid leukemia cells (American Type Culture Collection, Manassas, Va.) were added to 96-well clear bottom assay plates containing RPMI-1640 medium supplemented with 10% fetal bovine serum (FBS) at approximately 30,000 cells/well and incubated for 24 hours at 37° C. with 5% CO₂ and 95% humidity. Control wells containing no cells were included to measure background fluorescence signal. Test compounds were dissolved at 10-20 μM and diluted two-fold in DMSO to produce a working stock of compound solutions. Aliquots of the working stock solutions were subsequently diluted 100-fold in basal RPMI-1640 medium which was then further diluted 10-fold to the assay plate containing the cells to provide 10 test concentrations ranging from 0.04 μM-20 μM. After a 72-hour exposure, cell viability was measured using CellTiter-Blue® (Promega, Madison, Wis.) to calculate GI₅₀, the compound concentration resulting in 50% growth inhibition, using a four-parameter dose-response model.

Prior to generation of dose response curves, the data were background subtracted using the no cell control values (mean+/−standard deviation) and fluorescence values versus Log₁₀ concentration of test compounds were plotted using GraphPad Prism. The resulting sigmoidal curve was then fit to the graph and IC₅₀ values were calculated using a 4 parameter (4PL) algorithm using the following equation: 4(PL) F(x)=(A−D)/(1+(x/c)^{B}+D, where A=lower asymptote (baseline response), D=upper asymptote (maximum response), C=drug concentration that provokes a response halfway between A and D, B=slope of the curve.

Certain compounds of the present invention possessed GI₅₀ values of less than 1 μM, some compounds possessed GI₅₀ values between 1 μM and 20 μM, and other compounds possessed GI₅₀ values greater than 20 μM, indicating a range of potency for the compounds of the present invention toward inhibiting growth of MV4; 11 cells.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

### General Methods for Preparation of Compounds

The present invention also provides a method for the synthesis of compounds of the present invention. The present invention further provides a method for the synthesis of compounds useful as intermediates in the preparation of compounds of the present invention. The compounds can be prepared according to the methods described below using readily available starting materials and reagents. In these reactions, variants may be employed which are themselves known to those of ordinary skill in this art but are not described in detail here. Those skilled in the art of organic synthesis will appreciate that there exist multiple means of producing compounds of the present invention. Illustrative synthetic methods, including those directed to specific, selected compounds noted in Tables 1 and 2, are as set forth herein.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by one of ordinary skill in the art by routine optimization procedures.

One skilled in the art of organic synthesis understands that vulnerable moieties such as C(O)OH, C(O) and C(O)H, NH, C(O)NH2, OH and SH moieties may be protected and deprotected, as necessary. Protecting groups for C(O)OH moieties include, but are not limited to, allyl, benzoylmethyl, benzyl, benzyloxymethyl, tert-butyl, ethyl, methyl, 2,2,2-trichloroethyl, and the like. Protecting groups for C(O) and C(O)H moieties include, but are not limited to, 1,3-dioxylketal, diethylketal, dimethylketal, 1,3-dithianylketal, O-methyloxime, 0-phenyloxime and the like. Protecting groups for NH moieties include, but are not limited to, acetyl, benzoyl, benzyl (phenylmethyl), benzylidene, benzyloxycarbonyl (Cbz), tert-butoxycarbonyl (Boc), 3,4-dimethoxybenzyloxycarbonyl, diphenylmethyl, diphenylphosphoryl, formyl, methanesulfonyl, para-methoxybenzyloxycarbonyl, phenylacetyl, phthaloyl, succinyl, trichloroethoxycarbonyl, triethylsilyl, trifluoroacetyl, trimethylsilyl, triphenylmethyl, triphenylsilyl, para-toluenesulfonyl and the like.

Protecting groups for OH and SH moieties include, but are not limited to, acetyl, allyl, allyloxycarbonyl, benzyloxycarbonyl (Cbz), benzoyl, benzyl, tert-butyl, tert-butyldimethylsilyl, tert-butyldiphenylsilyl, 3,4-dimethoxybenzyl, 3,4-dimethoxybenzyloxycarbonyl, 1,1-dimethyl-2-propenyl, diphenylmethyl, methanesulfonyl, methoxyacetyl, 4-methoxybenzyloxycarbonyl, para-methoxybenzyl, methoxycarbonyl, methyl, para-toluenesulfonyl, 2,2,2-trichloroethoxycarbonyl, 2,2,2-trichloroethyl, triethylsilyl, trifluoroacetyl, 2-(trimethylsilyl)ethoxycarbonyl, 2-trimethylsilylethyl, triphenylmethyl, 2-(triphenylphosphonio)ethoxycarbonyl and the like.

A discussion of protecting groups is provided in T. H. Greene and P. G. M. Wuts, Protective Groups in Organic Synthesis, 3rd Ed., John Wiley & Sons, New York (1999).

### EXAMPLES

The invention will now be further described with reference to the following illustrative examples in which, unless stated otherwise: (i) temperatures are given in degrees Celsius (° C.); operations are carried out at room temperature (RT) or ambient temperature, that is, in a range of 18-25° C.; (ii) organic solutions were dried over anhydrous sodium or magnesium sulfate unless otherwise stated; evaporation of organic solvent was carried out using a rotary evaporator under reduced pressure; (iii) column chromatography means flash chromatography on silica gel; thin layer chromatography (TLC) was carried out on silica gel plates; (iv) in general, the course of reactions was followed by TLC or liquid chromatography/mass spectroscopy (LC/MS) and reaction times are given for illustration only; (v) final products have satisfactory proton nuclear magnetic resonance (NMR) spectra and/or mass spectra data; (vi) yields are given for illustration only and are not necessarily those which can be obtained by diligent process development; preparations were repeated if more material was required; (vii) when given, NMR data is in the form of delta values for major diagnostic protons, given in part per million (ppm) relative to tetramethylsilane (TMS) as an internal standard, and were obtained in the solvent indicated; (viii) chemical symbols have their usual meanings; (ix) in the event that the nomenclature assigned to a given compound does not correspond to the compound structure depicted herein, the structure will control; (x) solvent ratio is given in volume:volume (v/v) terms.

Compounds of Formulae I, II and Ill may be prepared according to Scheme 1 as set forth below. Bromoanilines (1 or 6), boronic acids (4), amines (10) and alkylating and acylating reagents (11) are either commercially available or can be prepared by known methods. The reaction of a bromoaniline (1) with a dialkyl malonate (2) under thermal conditions will produce a compound (3) which can be then treated with an appropriate alkyl or aryl or heteroaryl boronic acid (4) under Suzuki reaction conditions to yield a compound 5. Alternatively, compound 5 can be prepared by reacting an appropriately substituted aniline (6) with a dialkyl malonate (2) under thermal conditions. The compound (5) can subsequently be cyclized to produce a 4-hydroxyquinolin-2(1H)-one derivative (7) by heating with polyphosphoric acid (PPA). The reaction of a 4-hydroxyquinolin-2(1H)-one (7) with POCl₃ will produce the corresponding dichloro compound (8) and subsequent treatment of a compound (8) with aqueous HCl will generate a compound 9. The treatment of compound 9 with an appropriate amine, R₁-L₂-NH—R₂ (10), wherein each of the variables is as defined previously, will produce the corresponding amine (Formula II). A compound of Formula II can be further derivatized to yield compounds of Formula I by alkylation with a suitable species, X-L₃-R₆ (11), where L₃ and R₆ are as previously defined and X represents a leaving group, according to methods know to those skilled in the art.

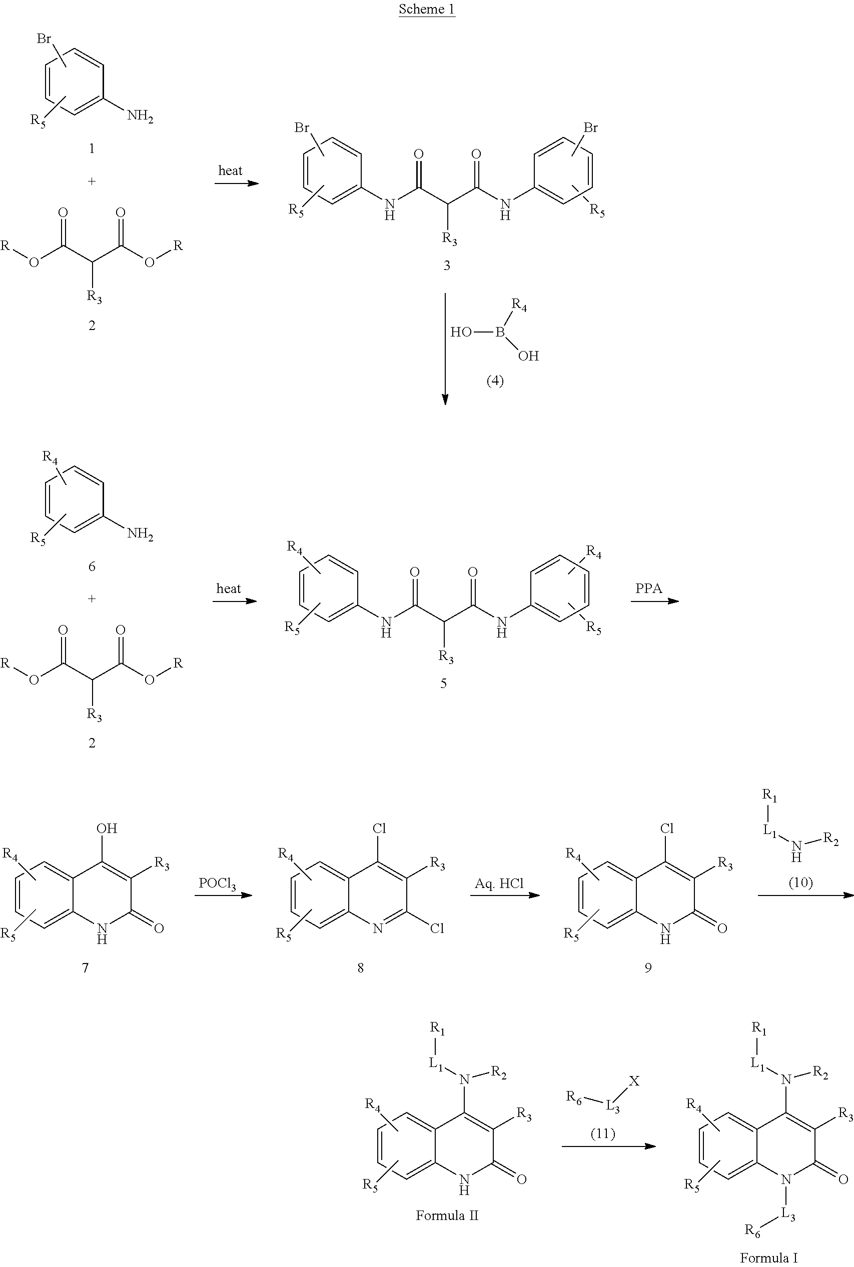

Scheme 2 illustrates an alternative synthetic route for preparation of compounds of the invention. A 6-bromo-4-chloroquinolin-2(1H)-one (12) can be prepared by following the synthetic steps to prepare compound 9 in Scheme 1. Compound 12 can be treated with methyl iodide to provide a compound 13, which can be then treated with appropriate alkyl or aryl or heteroaryl boronic acid (4) under Suzuki reaction conditions to yield a compound 14. The alkylation of compound 14 with an appropriate amine (10) will produce the corresponding amino derivative (Formula II) which can be further derivatized by treating with appropriate alkylating or acylating reagent (11) to yield compounds of Formula I.

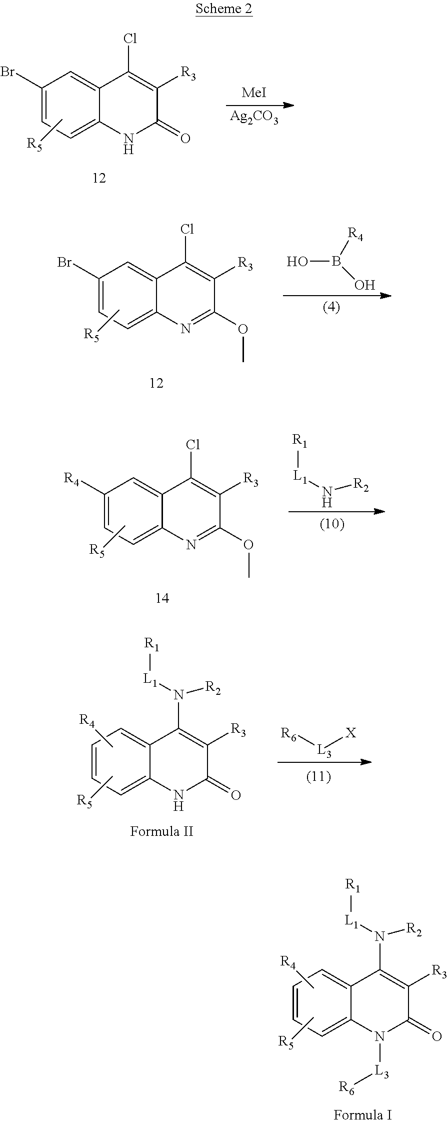

### SYNTHETIC EXAMPLES

Examples 1-4 may be prepared in similar fashion to Example 5 (below), substituting the appropriate hydrazine for the 1-methyl-1-phenylhydrazine and the appropriate 4-(4-chloro-2-methoxyquinoline for 4-(4-chloro-2-methoxyquinolin-6-yl)-3,5-dimethylisoxazole in Step 3.

### Example 5. 6-(3,5-dimethylisoxazol-4-yl)-4-(2-methyl-2-phenylhydrazinyl)-quinolin-2(1H)-one

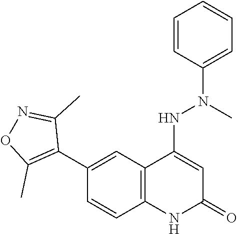 [SMILES: Cc1noc(C)c1-c1ccc2[nH]c(=O)cc(NN(C)c3ccccc3)c2c1]

### Step 1. 6-bromo-4-chloro-2-methoxyquinoline

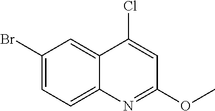

At 0° C., to a stirred solution of 6-bromo-4-chloroquinolin-2(1H)-one (1.8 g, 6.96 mmol) in DCM (20 mL) were added Ag₂CO₃ (1.92 g, 6.96 mmo) and MeI (1.09 g, 7.66 mmol). After stirred at r.t. overnight, the reaction mixture was quenched with H₂O. The layers were separated and the organic layer was washed with brine, dried over Na₂SO₄. Solvents were removed under vacuum and the residue was purified by flash chromatography (silica gel, 0˜30% ethyl acetate in petroleum ether) to provide 6-bromo-4-chloro-2-methoxyquinoline (2) (1.4 g) as a yellow solid. LC-MS (ESI): m/z (M/M+2) 272.1/274.1.

### Step 2. 4-(4-chloro-2-methoxyquinolin-6-yl)-3,5-dimethylisoxazole

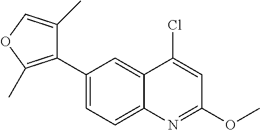 [SMILES: COc1cc(Cl)c2cc(-c3c(C)coc3C)ccc2n1]

At 90° C. under N₂ atmosphere, the mixture of 6-bromo-4-chloro-2-methoxyquinoline (1.4 g, 5.13 mmol), (3,5-dimethylisoxazol-4-yl)boronic acid (860 mg, 6.15 mmol) in MeCN/dioxane/H₂O (12/12/4 mL) was added K₂CO₃ (2.0 g, 15.38 mmol) and Pd(dppf)Cl₂.DCM (400 mg, 0.51 mmol). After stirred at 90° C. for 2 hr, the reaction mixture was cooled downed to rt, the reaction mixture was concentrated and purified by flash chromatography (silica gel, 0˜20% ethyl acetate in petroleum ether) to provide 4-(4-chloro-2-methoxyquinolin-6-yl)-3,5-dimethylisoxazole (4) (1.2 g, 81%) as a yellow oil. LC-MS (ESI): m/z (M/M+2) 289.2/291.2.

### Step 3. 4-(2-methoxy-4-(2-methyl-2-phenylhydrazinyl)quinolin-6-yl)-3,5-dimethylisoxazole

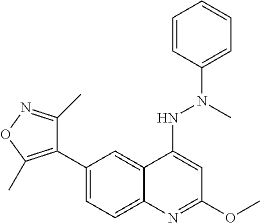 [SMILES: COc1cc(NN(C)c2ccccc2)c2cc(-c3c(C)noc3C)ccc2n1]

At 90° C. under N₂ atmosphere, the mixture of 4-(4-chloro-2-methoxyquinolin-6-yl)-3,5-dimethylisoxazole (110 mg, 0.38 mmol), 1-methyl-1-phenylhydrazine (53 mg, 0.42 mmol) in dioxane (1 mL) were added Pd₂(dba)₃ (35.2 mg, 0.038 mmol), xantphos (33 g, 0.057 mmol) and ^{t}BuOK (128 mg, 1.14 mmol). After stirred at 90° C. for 1 hr, the reaction mixture was cooled downed to rt, the reaction mixture was concentrated and purified by flash chromatography (silica gel, 0˜50% ethyl acetate in petroleum ether) to provide methyl 4-(2-methoxy-4-(2-methyl-2-phenylhydrazinyl)quinolin-6-yl)-3,5-dimethylisoxazole (4) (31 mg, 21%) as a yellow oil. LC-MS (ESI): m/z (M+1) 375.2.

### Step 4. 6-(3,5-dimethylisoxazol-4-yl)-4-(2-methyl-2-phenylhydrazinyl)quinolin-2(1H)-one

At 120° C. under N₂ atmosphere, to a stirred solution of methyl 4-(2-methoxy-4-(2-methyl-2-phenylhydrazinyl)quinolin-6-yl)-3,5-dimethylisoxazole (31 mg, 0.09 mmol) in DMF (0.5 mL) were added LiCI (36 mg, 0.9 mmol) and TsOH.H₂O (180 mg, 0.9 mmol). After stirred at 120° C. for 4 hr, the reaction mixture was cooled downed to r.t., and the reaction mixture was partitioned between EA and H₂O. The layers were separated and the organic layer was dried over Na₂SO₄. Solvents were removed under vacuum to and the residue was purified by R.P. HPLC (C18, 0˜90% acetonitrile in H₂O with 0.1% formic acid) to provide 6-(3,5-dimethylisoxazol-4-yl)-4-(2-methyl-2-phenylhydrazinyl)quinolin-2(1H)-one (10.7 mg, 36%) as a white solid. LC-MS (ESI): m/z (M+1) 361.3. 1H NMR (400 MHz, DMSO) δ 11.06 (s, 1H), 9.24 (s, 1H), 7.91 (s, 1H), 7.52 (dd, J=8.5, 1.5 Hz, 1H), 7.38 (d, J=8.5 Hz, 1H), 7.29-7.21 (m, 2H), 6.88 (d, J=8.0 Hz, 2H), 6.84-6.79 (m, 1H), 5.51 (s, 1H), 3.19 (s, 3H), 2.43 (s, 3H), 2.27 (s, 3H).

Examples 6-107 may be prepared in similar fashion to Example 5, substituting the appropriate hydrazine and chloroquinoline.

### Example 108. 4-(3-chlorobenzylamino)-6-(3,5-dimethylisoxazol-4-yl)quinolin-2(1H)-one)

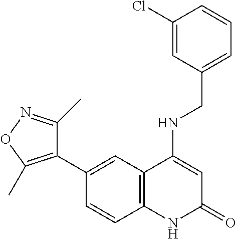 [SMILES: Cc1noc(C)c1-c1ccc2[nH]c(=O)cc(NCc3cccc(Cl)c3)c2c1]

4-(3-Chlorobenzylamino)-6-(3,5-dimethylisoxazol-4-yl)quinolin-2(1H)-one (7 mg, 23%) was obtained as a white solid following a similar procedure outlined in Example 170, from 6-bromo-4-(3-chlorobenzylamino)quinolin-2(1H)-one (30 mg, 0.08 mmol) and 3,5-dimethylisoxazol-4-ylboronic acid (23 mg, 0.16 mmol). LC-MS (ESI): m/z (M+1) 380.3. ¹H NMR (400 MHz, DMSO) δ 10.89 (s, 1H), 7.97 (s, 1H), 7.70 (t, J=6.0 Hz, 1H), 7.48 (dd, J=8.4, 1.6 Hz, 1H), 7.43 (s, 1H), 7.41-7.30 (m, 4H), 5.19 (s, 1H), 4.48 (d, J=5.8 Hz, 2H), 2.44 (s, 3H), 2.27 (s, 3H).

Examples 109-155 may be prepared in similar fashion to Example 108.

### Example 156. (R)-6-(3,5-dimethylisoxazol-4-yl)-44(1-phenylethyl)amino)quinolin-2(1H)-one

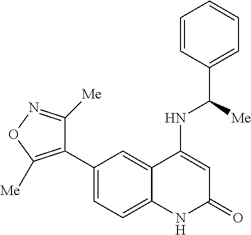 [SMILES: Cc1noc(C)c1-c1ccc2[nH]c(=O)cc(N[C@H](C)c3ccccc3)c2c1]

Following a similar procedure as in Example 170, but substituting benzylamine with (R)-(+)-α-methylbenzylamine, the desired product was obtained as a cream solid (11 mg). 6H (DMSO-d6, 400 MHz) 10.85 (s, 1H, NH), 8.22 (s, 1H, Ar), 7.45 (d, 1H, J=8.4, Ar), 7.42-7.27 (m, 5H, Ar), 7.24 (t, 1H, J=6.4, NH), 7.14 (d, 1H, J=8.4, Ar), 5.09 (s, 1H, CH), 4.67 (t, 1H, J=6.4, CH), 2.43 (s, 3H, Me), 2.27 (s, 3H, Me), 1.56 (d, 3H, J=6.4, CHMe).

Examples 157-159 may be prepared in similar fashion to Example 156.

### Example 160. 4-(benzylamino)-6-(3,5-di methyl isoxazol-4-yl)-N,N-dimethyl-2-oxo-1,2-dihydroquinoline-7-carboxamide

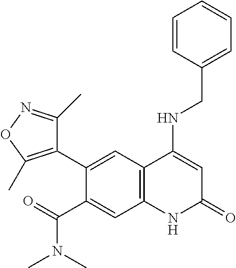 [SMILES: Cc1noc(C)c1-c1cc2c(NCc3ccccc3)cc(=O)[nH]c2cc1C(=O)N(C)C]

### Step 1. Methyl 5-amino-2-bromobenzoate (1)

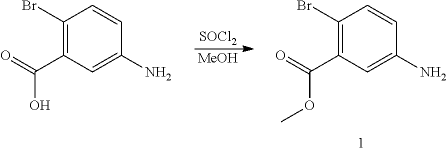

To a stirred solution of 5-amino-2-bromobenzoic acid (10.8 g, 50 mmol) in MeOH (50 mL) was added SOCl₂ (18 mL). After stirred at 50° C. overnight, the reaction mixture was concentrated and partitioned between ethyl acetate (50 mL) and H₂O (50 mL). The layers were separated and the organic layer was washed with sat. NaHCO₃ and brine, dried over Na₂SO₄. Solvents were removed under vacuum and the obtained crude product methyl 5-amino-2-bromobenzoate (1) (11.5 g, 100%) was used in the next step without purification. LC-MS (ESI): m/z (M/M+2) 230.1/232.1.

### Step 2. Methyl 2-bromo-5-(3-ethoxy-3-oxopropanamido)benzoate (2)

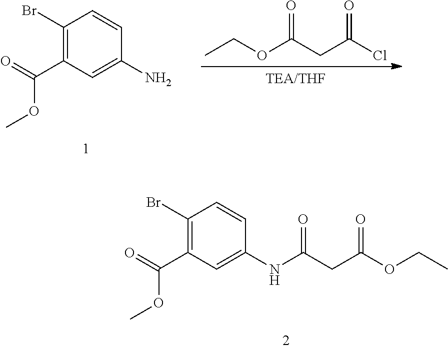

To a solution of methyl 5-amino-2-bromobenzoate (11.5 g, 50 mmol) and TEA (20.5 mL, 150 mmol) in THF (150 mL) was added ethyl 3-chloro-3-oxopropanoate (11.3 g, 75 mmol) at 0° C. After stirred at room temperature overnight, the reaction mixture was quenched with sat.NaHCO₃, the layers were separated and the aqueous layer was extracted with ethylacetate (50 mL×3). The combined organic layers were washed with brine, dried over Na₂SO₄. Solvents were removed under vacuum and the obtained crude product methyl 2-bromo-5-(3-ethoxy-3-oxopropanamido)benzoate (2) (12.9 g, 75%) was used in the next step without purification. LC-MS (ESI): m/z (M/M+1) 344.0/346.0.

### Step 3. Methyl 6-bromo-4-hydroxy-2-oxo-1,2-dihydroquinoline-7-carboxylate (3)

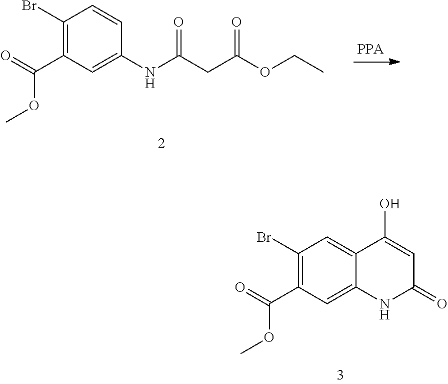 [SMILES: CCOC(=O)CC(=O)Nc1ccc(Br)c(C(=O)OC)c1 ; COC(=O)c1cc2[nH]c(=O)cc(O)c2cc1Br]

A mixture of methyl 2-bromo-5-(3-ethoxy-3-oxopropanamido)benzoate (2) (5 g, 14.5 mmol) in PPA (24.5 g) was stirred at 130° C. for 8 hr. After cooled downed to room temperature, the reaction mixture was poured into ice water and the newly generated yellow solid was collected by filtration to provide methyl 6-bromo-4-hydroxy-2-oxo-1,2-dihydroquinoline-7-carboxylate (3) (5 g, quant. yield) which was used in the next step without purification. LC-MS (ESI): m/z (M/M+2) 298.1/300.1.

### Step 4. Methyl 6-bromo-2,4-dichloroquinoline-7-carboxylate (4)

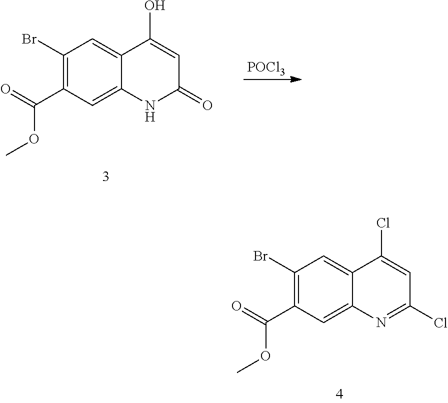

A mixture of methyl 6-bromo-4-hydroxy-2-oxo-1,2-dihydroquinoline-7-carboxylate (3) and its isomer (4.3 g, 14.5 mmol) in POCl₃ (25 mL) was stirred at 130° C. for 8 hrs. After cooled downed to room temperature, the reaction mixture was poured into ice water and extracted with ethyl acetate (100 mL). The organic layer was dried over Na₂SO₄. Solvents were removed under vacuum and the residue was purified by flash chromatography (silica gel, 0˜50% ethyl acetate in petroleum ether) to provide methyl 6-bromo-2,4-dichloroquinoline-7-carboxylate (4) (238 mg, 5%) as a yellow solid. LC-MS (ESI): m/z (M/M+2) 334.0/336.0.

### Step 5. Methyl 6-bromo-4-chloro-2-oxo-1,2-dihydroquinoline-7-carboxylate (5)

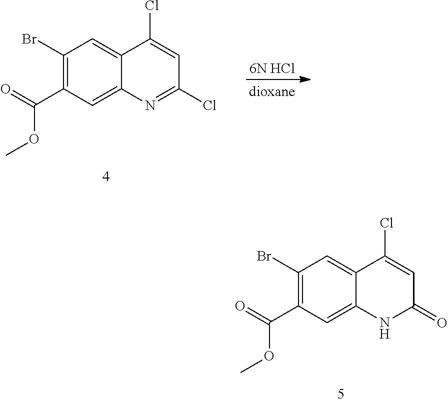

To a suspension of 6-bromo-2,4-dichloroquinoline-7-carboxylate (4) (238 mg, 0.71 mmol) in dioxane (5 mL) was added 6N HCl (5 mL). After stirred at 100° C. overnight, the reaction mixture was cooled down to room temperature and extracted with ethylacetate (30 mL). The organic layer was dried over Na₂SO₄. Solvents were removed under vacuum and the residue was purified by flash chromatography (silica gel, 0˜50% ethyl acetate in petroleum ether) to provide methyl 6-bromo-4-chloro-2-oxo-1,2-dihydroquinoline-7-carboxylate (5) (128 mg, 57%) as a yellow solid. LC-MS (ESI): m/z (M/M+2) 316.0/318.0.

### Step 6. Methyl 4-(benzylamino)-6-bromo-2-oxo-1,2-dihydroquinoline-7-carboxylate (6)

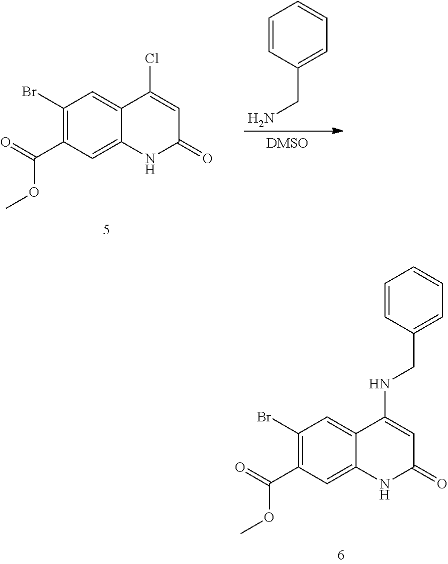 [SMILES: COC(=O)c1cc2[nH]c(=O)cc(Cl)c2cc1Br ; NCc1ccccc1 ; COC(=O)c1cc2[nH]c(=O)cc(NCc3ccccc3)c2cc1Br]

To the solution of methyl 6-bromo-4-chloro-2-oxo-1,2-dihydroquinoline-7-carboxylate (5) (128 mg, 0.4 mmol) in DMSO (2 mL) was added benzyl amine (86 mg, 0.8 mmol). After stirred at 120° C. overnight, the reaction mixture was cooled down to room temperature, diluted with water and extracted with ethylacetate (20 mL). The organic layer was dried over Na₂SO₄. Solvents were removed under vacuum and the residue was purified by flash chromatography (silica gel, 0˜80% ethyl acetate in petroleum ether) to provide methyl 4-(benzylamino)-6-bromo-2-oxo-1,2-dihydroquinoline-7-carboxylate (6) (150 mg, 97%) as a yellow solid. LC-MS (ESI): m/z (M/M+2) 387.0/389.0.

### Step 7. Methyl 4-(benzylamino)-6-(3,5-dimethylisoxazol-4-yl)-2-oxo-1,2-dihydroquinoline-7-carboxylate (7)

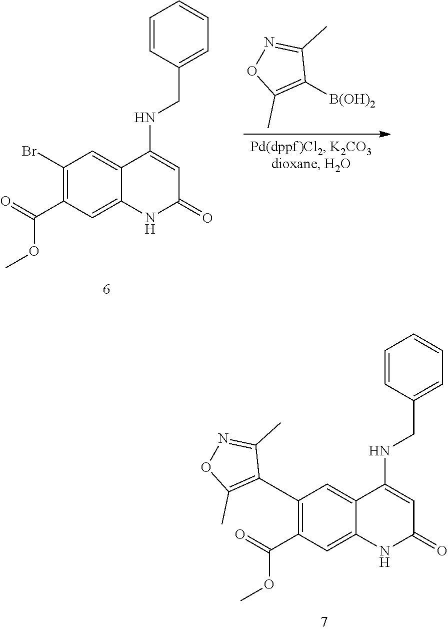

Under N₂ atmosphere, to a solution of methyl 4-(benzylamino)-6-bromo-2-oxo-1,2-dihydroquinoline-7-carboxylate (6) (150 mg, 0.39 mmol) in dioxane/H₂O (2 mL/0.05 mL) were added Pd(dppf)Cl₂DCM (32 mg, 0.039 mmol), K₂OO₃ (161 mg, 1.17 mmol) and 3,5-dimethylisoxazol-4-ylboronic acid (71 mg, 0.5 mmol). After stirred at 100° C. overnight, the reaction mixture was concentrated down and purified by flash chromatography (silica gel, 0˜80% ethyl acetate in petroleum ether) to provide methyl 4-(benzylamino)-6-(3,5-dimethylisoxazol-4-yl)-2-oxo-1,2-dihydroquinoline-7-carboxylate (7) (109 mg, 69%) as a yellow solid. LC-MS (ESI): m/z (M+1) 404.4.

### Step 8. 4-(benzylamino)-6-(3,5-dimethylisoxazol-4-yl)-2-oxo-1,2-dihydroquinoline-7-carboxylic acid (8)

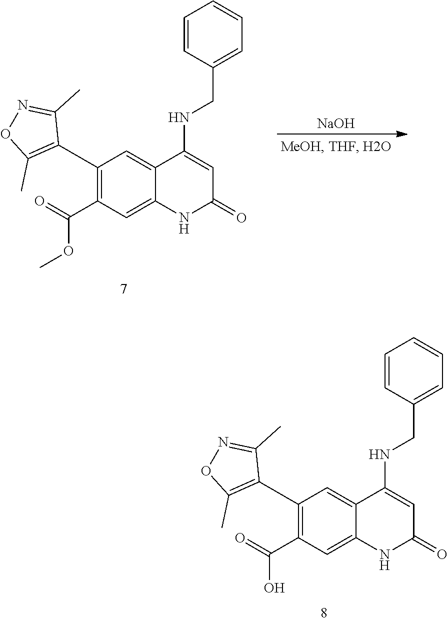 [SMILES: COC(=O)c1cc2[nH]c(=O)cc(NCc3ccccc3)c2cc1-c1c(C)noc1C ; "NaOH" ; Cc1noc(C)c1-c1cc2c(NCc3ccccc3)cc(=O)[nH]c2cc1C(=O)O]

To a solution of methyl 4-(benzylamino)-6-(3,5-dimethylisoxazol-4-yl)-2-oxo-1,2-dihydroquinoline-7-carboxylate (7) (109 mg, 0.09 mmol) in MeOH/THF/H₂O (2 mL/2 mL/0.5 mL) was added NaOH (11 mg, 0.27 mmol). After stirred at room temperature overnight, the reaction mixture was acidified to pH-1 by 1N HCl. Extracted with 85% DCM/IPA (10 mL*3) and dried over Na₂SO₄. Solvents were removed under vacuum and the residue 4-(benzylamino)-6-(3,5-dimethylisoxazol-4-yl)-2-oxo-1,2-dihydroquinoline-7-carboxylic acid (8) (105 mg, quant. yield) was used in the next step without purification LC-MS (ESI): m/z (M+1) 390.2.

### Step 9. 4-(benzylamino)-6-(3,5-dimethylisoxazol-4-yl)-N,N-dimethyl-2-oxo-1,2-dihydroquinoline-7-carboxamide

To a solution of 4-(benzylamino)-6-(3,5-dimethylisoxazol-4-yl)-2-oxo-1,2-dihydro quinoline-7-carboxylic acid (8) (36 mg, 0.09 mmol) in DMF (2 mL) were added dimethylamine HCl salt (9 mg, 0.11 mmol), DIEA (36 mg, 0.27 mmol) and HBTU (51 mg, 0.135 mmol). After stirred at room temperature overnight, the reaction mixture was quenched with sat. NaHCO₃ and extracted with ethylacetate (10 mL). The organic layer was dried over Na₂SO₄. Solvents were removed under vacuum and the residue was purified by prep. HPLC (C18, 0-90% acetonitrile in H₂O with 0.1% formic acid) to provide 4-(benzylamino)-6-(3,5-dimethylisoxazol-4-yl)-N,N-dimethyl-2-oxo-1,2-dihydroquinoline-7-carboxamide T1-53 (9) (7 mg, 19%) as a white solid. LC-MS (ESI): m/z (M+1) 417.2. ¹H NMR (400 MHz, DMSO) δ 10.98 (s, 1H), 8.00 (s, 1H), 7.77-7.71 (m, 1H), 7.40-7.31 (m, 4H), 7.28-7.23 (m, 1H), 7.17 (s, 1H), 5.21 (s, 1H), 4.44 (d, J=5.6 Hz, 2H), 2.84 (s, 3H), 2.63 (s, 3H), 2.27 (s, 3H), 2.12 (s, 3H).

### Example 161: 4-(benzylamino)-6-(3,5-dimethylisoxazol-4-yl)-2-oxo-1,2-dihydroquinoline-7-carboxamide

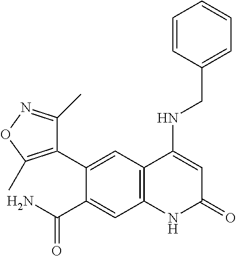 [SMILES: Cc1noc(C)c1-c1cc2c(NCc3ccccc3)cc(=O)[nH]c2cc1C(N)=O]

4-(Benzylamino)-6-(3,5-dimethylisoxazol-4-yl)-2-oxo-1,2-dihydroquinoline-7-carboxamide (4 mg, 11%) was obtained as a white solid following a similar procedure outlined in Example 160, via 4-(benzylamino)-6-(3,5-dimethylisoxazol-4-yl)-2-oxo-1,2-dihydroquinoline-7-carboxylic acid (35 mg, 0.09 mmol) and NH₄Cl (15 mg, 0.27 mmol). LC-MS (ESI): m/z (M+1) 389.3. ¹H NMR (400 MHz, DMSO) δ 10.97 (s, 1H), 7.94 (s, 1H), 7.72-7.67 (m, 2H), 7.41-7.32 (m, 6H), 7.29-7.23 (m, 1H), 5.21 (s, 1H), 4.43 (d, J=5.6 Hz, 2H), 2.27 (s, 3H), 2.11 (s, 3H).

### Example 162. 4-(benzylamino)-6,7-bis(3,5-dimethylisoxazol-4-yl)quinolin-2(1H)-one

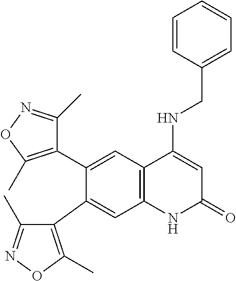 [SMILES: Cc1noc(C)c1-c1cc2[nH]c(=O)cc(NCc3ccccc3)c2cc1-c1c(C)noc1C]

### Step 1. 4-(benzylamino)-6,7-dibromoquinolin-2(1H)-one

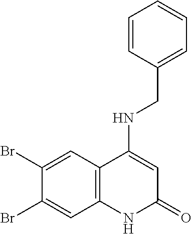 [SMILES: O=c1cc(NCc2ccccc2)c2cc(Br)c(Br)cc2[nH]1]

4-(Benzylamino)-6,7-dibromoquinolin-2(1H)-one (20 mg, 24%) was obtained as a white solid following a similar procedure to that outlined in Example 160, starting from 3,4-dibromoaniline (3.5 g, 14 mmol) and ethyl 3-chloro-3-oxopropanoate (3.2 g, 21 mmol). LC-MS (ESI): m/z (M/M+2/M+4)=406.9/408.9/410.9.

### Step 2. 4-(benzylamino)-6,7-bis(3,5-dimethylisoxazol-4-yl)quinolin-2(1H)-one

4-(Benzylamino)-6,7-bis(3,5-dimethylisoxazol-4-yl)quinolin-2(1H)-one (9 mg, 40%) was obtained as a white solid following a similar procedure outlined in Example 160, starting from 4-(benzylamino)-6,7-dibromoquinolin-2(1H)-one (8a, 20 mg, 0.05 mmol) and 3,5-dimethylisoxazol-4-ylboronic acid (18 mg, 0.125 mmol). LC-MS (ESI): m/z (M+1) 441.2. ¹H NMR (400 MHz, DMSO) δ 10.85 (s, 1H), 8.09 (s, 1H), 7.81-7.75 (m, 1H), 7.40-7.33 (m, 4H), 7.30-7.23 (m, 1H), 7.21 (s, 1H), 5.21 (s, 1H), 4.47 (d, J=5.6 Hz, 2H), 2.18-1.85 (m, 12H).

### Example 163. 4-(3-chlorobenzylamino)-6-(pyridin-3-yl)quinolin-2(1H)-one

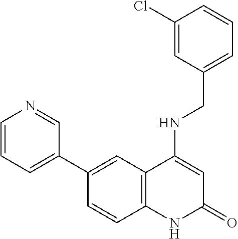 [SMILES: O=c1cc(NCc2cccc(Cl)c2)c2cc(-c3cccnc3)ccc2[nH]1]

### Step 1. 6-bromo-4-(3-chlorobenzylamino)quinolin-2(1H)-one

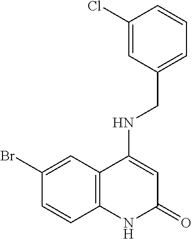 [SMILES: O=c1cc(NCc2cccc(Cl)c2)c2cc(Br)ccc2[nH]1]

6-Bromo-4-(3-chlorobenzylamino)quinolin-2(1H)-one (1.3 g, 40%) was obtained as a yellow solid following a similar procedure to those presented above, starting from 4-dibromoaniline (10 g, 60 mmol) and ethyl 3-chloro-3-oxopropanoate (10.8 g, 72 mmol). LC-MS (ESI): m/z (M/M+2) 381.2/383.2.

### Step 2. 4-(3-chlorobenzylamino)-6-(pyridin-3-yl)quinolin-2(1H)-one

4-(3-Chlorobenzylamino)-6-(pyridin-3-yl)quinolin-2(1H)-one (7 mg, 16%) was obtained as a white solid following a similar procedure outlined above, starting from 6-bromo-4-(3-chlorobenzylamino)quinolin-2(1H)-one (70 mg, 0.12 mmol) and pyridin-3-ylboronic acid (47 mg, 0.24 mmol). LC-MS (ESI): m/z (M+1) 362.4. ¹H NMR (400 MHz, DMSO) δ 10.91 (s, 1H), 9.04 (d, J=2.0 Hz, 1H), 8.57 (dd, J=4.7, 1.4 Hz, 1H), 8.43 (s, 1H), 8.20-8.14 (m, 1H), 7.92-7.83 (m, 2H), 7.52 (dd, J=7.9, 4.8 Hz, 1H), 7.46 (s, 1H), 7.43-7.31 (m, 4H), 5.21 (s, 1H), 4.51 (d, J=5.7 Hz, 2H).

### Example 164. 4-(3-chlorobenzylamino)-6-(5-methoxypyridin-3-yl)quinolin-2(1H)-one

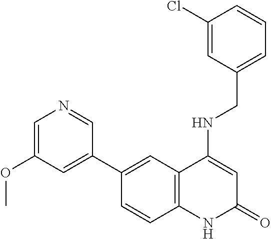 [SMILES: COc1cncc(-c2ccc3[nH]c(=O)cc(NCc4cccc(Cl)c4)c3c2)c1]

4-(3-Chlorobenzylamino)-6-(5-methoxypyridin-3-yl)quinolin-2(1H)-one (13 mg, 24%) was obtained as a white solid following a similar procedure to those outlined above, starting from 6-bromo-4-(3-chlorobenzylamino)quinolin-2(1H)-one (50 mg, 0.14 mmol) and 3-methoxy-5-(4,4,5,5-tetramethyl-1,3,2-dioxaborolan-2-yl) pyridine (70 mg, 0.28 mmol). LC-MS (ESI): m/z (M+1) 392.4. ¹H NMR (400 MHz, DMSO) δ 10.91 (s, 1H), 8.65 (d, J=1.7 Hz, 1H), 8.41 (s, 1H), 8.31 (d, J=2.7 Hz, 1H), 7.91 (dd, J=8.6, 1.7 Hz, 1H), 7.87-7.82 (m, 1H), 7.74-7.71 (m, 1H), 7.46 (s, 1H), 7.41-7.31 (m, 4H), 5.20 (s, 1H), 4.51 (d, J=5.7 Hz, 2H), 3.94 (s, 3H).

### Example 165. 4-(3-chlorobenzylamino)-6-(2-methoxypyridin-3-yl)quinolin-2(1H)-one

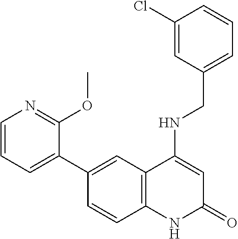 [SMILES: COc1ncccc1-c1ccc2[nH]c(=O)cc(NCc3cccc(Cl)c3)c2c1]

4-(3-Chlorobenzylamino)-6-(2-methoxypyridin-3-yl)quinolin-2(1H)-one (13 mg, 18%) was obtained as a white solid following a similar procedure outlined above, starting from 6-bromo-4-(3-chlorobenzylamino)quinolin-2(1H)-one (70 mg, 0.19 mmol) and 2-methoxypyridin-3-ylboronic acid (59 mg, 0.38 mmol). LC-MS (ESI): m/z (M+1) 392.4. ¹H NMR (400 MHz, DMSO) δ 10.85 (s, 1H), 8.20 (d, J=3.2 Hz, 1H), 8.16 (s, 1H), 7.86-7.82 (m, 1H), 7.76-7.67 (m, 2H), 7.44 (s, 1H), 7.42-7.30 (m, 3H), 7.27 (d, J=8.6 Hz, 1H), 7.14 (dd, J=7.2, 5.0 Hz, 1H), 5.18 (s, 1H), 4.47 (d, J=5.9 Hz, 2H), 3.91 (s, 3H).

### Example 166: 443-chlorobenzylamino)-6-(pyridin-4-yl)quinolin-2(1H)-one

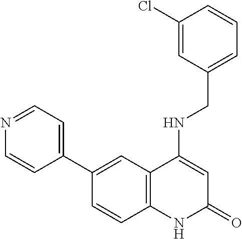 [SMILES: O=c1cc(NCc2cccc(Cl)c2)c2cc(-c3ccncc3)ccc2[nH]1]

4-(3-Chlorobenzylamino)-6-(pyridin-4-yl)quinolin-2(1H)-one (7 mg, 10%) was obtained as a white solid following a similar procedure to Example 170, from 6-bromo-4-(3-chlorobenzylamino)quinolin-2(1H)-one (70 mg, 0.19 mmol) and pyridin-4-ylboronic acid (95 mg, 0.77 mmol). LC-MS (ESI): m/z (M+1) 362.4. ¹H NMR (400 MHz, DMSO) δ 10.97 (s, 1H), 8.66 (d, J=5.5 Hz, 2H), 8.52 (s, 1H), 7.99-7.95 (m, 1H), 7.94-7.88 (m, 1H), 7.83 (d, J=6.1 Hz, 2H), 7.46 (s, 1H), 7.42-7.31 (m, 4H), 5.21 (s, 1H), 4.52 (d, J=5.7 Hz, 2H).

### Example 167. 4-(3-chlorobenzylamino)-6-(1-methyl-1H-pyrazol-4-yl)quinolin-2(1H)-one

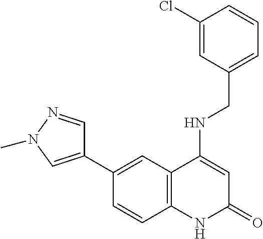 [SMILES: Cn1cc(-c2ccc3[nH]c(=O)cc(NCc4cccc(Cl)c4)c3c2)cn1]

4-(3-Chlorobenzylamino)-6-(1-methyl-1H-pyrazol-4-yl)quinolin-2(1H)-one (8 mg, 27%) was obtained as a white solid following a similar procedure outlined in Example 170, from 6-bromo-4-(3-chlorobenzylamino)quinolin-2(1H)-one (30 mg, 0.08 mmol) and 1-methyl-1H-pyrazol-4-ylboronic acid (21 mg, 0.16 mmol). LC-MS (ESI): m/z (M+1) 365.3. ¹H NMR (400 MHz, DMSO) δ 10.76 (s, 1H), 8.22 (s, 1H), 8.09 (s, 1H), 7.90 (s, 1H), 7.71-7.65 (m, 2H), 7.44 (s, 1H), 7.43-7.31 (m, 3H), 7.21 (d, J=8.5 Hz, 1H), 5.16 (s, 1H), 4.51 (d, J=5.8 Hz, 2H), 3.89 (s, 3H).

### Example 168. 4-(3-chlorobenzylamino)-6-phenylquinolin-2(1H)-one

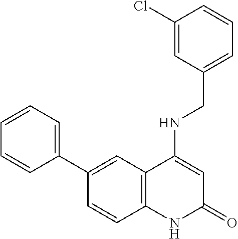 [SMILES: O=c1cc(NCc2cccc(Cl)c2)c2cc(-c3ccccc3)ccc2[nH]1]

4-(3-Chlorobenzylamino)-6-phenylquinolin-2(1H)-one (7 mg, 28%) was obtained as a white solid following a similar procedure outlined in Example 170, from 6-bromo-4-(3-chlorobenzylamino)quinolin-2(1H)-one (25 mg, 0.07 mmol) and phenylboronic acid (17 mg, 0.14 mmol). LC-MS (ESI): m/z (M+1) 361.2. ¹H NMR (400 MHz, DMSO) δ 10.86 (s, 1H), 8.35 (s, 1H), 7.90-7.76 (m, 4H), 7.50 (t, J=7.7 Hz, 2H), 7.45 (s, 1H), 7.42-7.30 (m, 5H), 5.19 (s, 1H), 4.50 (d, J=5.7 Hz, 2H).

### Example 169. 6-(3,5-dimethylisoxazol-4-yl)-8-fluoro-4-(2-fluorobenzylamino)quinolin-2(1H)-one

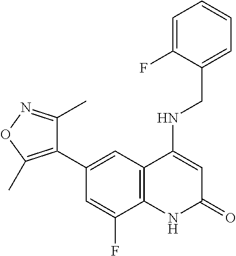 [SMILES: Cc1noc(C)c1-c1cc(F)c2[nH]c(=O)cc(NCc3ccccc3F)c2c1]

### Step 1. 6-Bromo-4-(3-chlorobenzylamino)-8-fluoroquinolin-2(1H)-one

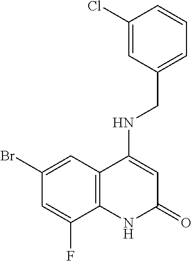 [SMILES: O=c1cc(NCc2cccc(Cl)c2)c2cc(Br)cc(F)c2[nH]1]

6-Bromo-4-(3-chlorobenzylamino)-8-fluoroquinolin-2(1H)-one (1.35 g) was obtained as a yellow solid from 4-bromo-2-fluoroaniline (5.0 g, 26.3 mmol) and ethyl 3-chloro-3-oxopropanoate (4.7 g, 31.6 mmol). LC-MS (ESI): m/z (M/M+2) 381.2/383.2.

### Step 2. 6-(3,5-Dimethylisoxazol-4-yl)-8-fluoro-4-(2-fluorobenzylamino)quinolin-2(1H)-one

6-(3,5-Dimethylisoxazol-4-yl)-8-fluoro-4-(2-fluorobenzylamino)quinolin-2(1H)-one (12 mg, 20%) was obtained as a white solid from 6-bromo-4-(3-chlorobenzylamino)-8-fluoroquinolin-2(1H)-one (60 mg, 0.16 mmol) and 3,5-dimethylisoxazol-4-ylboronic acid (33 mg, 0.24 mmol). LC-MS (ESI): m/z (M/M+2) 398.5/400.5. 1H NMR (400 MHz, DMSO) δ 10.84 (s, 1H), 7.83 (s, 1H), 7.80-7.75 (m, 1H), 7.49 (d, J=11.5 Hz, 1H), 7.44 (s, 1H), 7.42-7.31 (m, 3H), 5.25 (s, 1H), 4.49 (d, J=5.8 Hz, 2H), 2.45 (s, 3H), 2.29 (s, 3H).

### Example 170. 4-(benzylamino)-6-(3,5-dimethylisoxazol-4-yl)quinolin-2(1H)-one

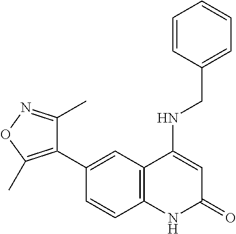 [SMILES: Cc1noc(C)c1-c1ccc2[nH]c(=O)cc(NCc3ccccc3)c2c1]

### Step 1. N1,N3-bis(4-bromophenyl)malonamide

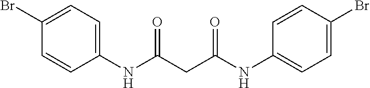 [SMILES: O=C(CC(=O)Nc1ccc(Br)cc1)Nc1ccc(Br)cc1]

4-Bromoaniline (20 mmol, 3.44 g, 2 eq.) and diethylmalonate (10 mmol, 1.60 g, 1 eq.) was heated to 150° C. for 20 hr. Reaction was cooled and diluted with ethanol and filtered to give product as a grey solid (1.10 g, TLC. 100% EtOAc). δH (DMSO-d6, 400 MHz) 10.32 (s, 2H, 2×NH2), 7.58 (d, 4H, J=9.6, Ar), 7.51 (d, 4H, J=9.6, Ar), 3.48 (s, 2H, CH2); 50 (DMSO-d6, 100 MHz) 165.9, 138.7, 132.0, 121.4, 115.4, 46.4.

### Step 2. N1,N3-bis(4-(3,5-dimethylisoxazol-4-yl)phenyl)malonamide

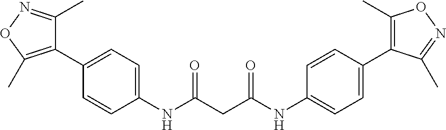 [SMILES: Cc1noc(C)c1-c1ccc(NC(=O)CC(=O)Nc2ccc(-c3c(C)noc3C)cc2)cc1]

Compound from Step 1 (0.50 mmol) and isoxazole boronic acid (1.08 mmol, 2.2 eq.) were dissolved in toluene/EtOH (8 mL/8:2). 2M Na₂CO₃ (1.47 mmol, 735 uL, 3 eq.) and palladium tetrakis (0.098 mmol, 113 mg, 20 mol %) were added and heated to 90° C. for 5 hr. Reaction was cooled, partitioned between EtOAc and H₂O. The organic potion was washed with H₂O, sat. NaCl and dried over Na₂SO₄. Gradient column chromatography gave product as a yellow solid (117 mg, TLC. 1:1/Hex:EtOAc). δH (CDCl3, 400 MHz) 9.25 (s, 2H, 2×NH2), 7.66 (d, 4H, J=8.6, Ar), 7.23 (d, 4H, J=8.6, Ar), 3.61 (s, 2H, CH2), 2.39 (s, 3H, Me), 2.56 (s, 3H, Me).

### Step 3. Preparation of 6-(3,5-dimethylisoxazol-4-yl)-4-hydroxyquinolin-2(1H)-one

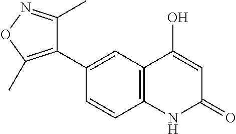 [SMILES: Cc1noc(C)c1-c1ccc2[nH]c(=O)cc(O)c2c1]

Compound from Step 2 (117 mg) was treated with polyphosphoric acid (585 mg, 5 eq. by weight) and heated to 140° C. for 5 hr. Reaction was cooled, diluted with H₂O, sonicated and filtered to give the desired product as a white solid (65 mg, TLC. 5% MeOH/EtOAc). δH (DMSO-d6, 400 MHz) 11.43 (br s, 1H, OH), 11.30 (s, 1H, NH), 7.70 (s, 1H, Ar), 7.51 (dd, 1H, J=8.4, 1.6, Ar), 7.35 (d, 1H, J=8.4, Ar), 5.77 (s, 1H, CH), 2.40 (s, 3H, Me), 2.22 (s, 3H, Me).

### Step 4. Preparation of 4-Chloro-6-(3,5-dimethylisoxazol-4-yl)quinolin-2(1H)-one

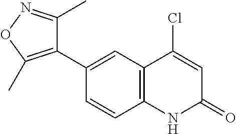 [SMILES: Cc1noc(C)c1-c1ccc2[nH]c(=O)cc(Cl)c2c1]

Compound Step 3 (0.156 mmol, 40 mg) was treated with NEt₃ (0.469 mmol, 65 uL, 3 eq.), POCl₃ (0.5 mL) and heated to 65° C. for 3 hr. Reaction was cooled, partitioned between EtOAc and H₂O. The organic potion was washed with H₂O, sat. NaCl and dried over Na₂SO₄. Gradient column chromatography gave the desired intermediate 4-(2,4-dichloroquinolin-6-yl)-3,5-dimethylisoxazole as a brown solid (TLC. 8:2/Hex:EtOAc). The solid was dissolved in dioxane (2 mL) and 6M HCl (2 mL) was added and refluxed for 4 hr. Reaction was cooled, diluted with H₂O, neutralized to pH 9 with solid K₂CO₃ and filtered to give the desired product as a cream solid (34 mg, 1:1/Hex:EtOAc). δH (DMSO-d6, 400 MHz) 12.15 (s, 1H, NH), 7.78 (s, 1H, Ar), 7.67 (d, 1H, J=8.4, Ar), 7.48 (d, 1H, J=8.4, Ar), 6.89 (s, 1H, CH), 2.42 (s, 3H, Me), 2.24 (s, 3H, Me).

### Step 5. 4-(benzylamino)-6-(3,5-dimethylisoxazol-4-yl)quinolin-2(1H)-one

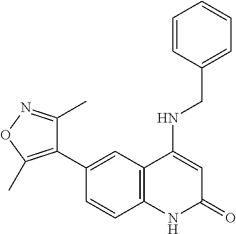 [SMILES: Cc1noc(C)c1-c1ccc2[nH]c(=O)cc(NCc3ccccc3)c2c1]

Compound from Step 4 (0.036 mmol, 10 mg) was heated to 120° C. in a 1:1 mixture of DMSO and 2-Cl-benzylamine (1 mL) overnight to 48 hr. Reaction was cooled, partitioned between EtOAc and H₂O. The organic potion was washed with H₂O, sat. NaCl and dried over Na₂SO₄. Gradient column chromatography gave the desired product after lyophilization as a cream solid (10 mg, TLC. 5% MeOH/DCM). δH (DMSO-d6, 400 MHz) 10.86 (s, 1H, NH), 7.99 (s, 1H, Ar), 7.71 (t, 1H, J=5.2, NH), 7.56 (d, 1H, J=8.8, Ar), 7.40-7.21 (m, 6H, Ar), 5.16 (s, 1H, CH), 4.46 (d, 2H, J=5.2, CH2), 2.42 (s, 3H, Me), 2.26 (s, 3H, Me).

### Example 171. 6-(3,5-dimethylisoxazol-4-yl)-44(2-methoxybenzyl)amino)quinolin-2(1H)-one

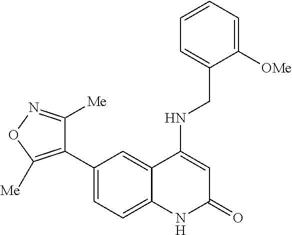 [SMILES: COc1ccccc1CNc1cc(=O)[nH]c2ccc(-c3c(C)noc3C)cc12]

Following a similar procedure as in Example 170, but substituting benzylamine with 2-OMe-benzylamine in Step 5, the desired product was obtained as a cream solid (7 mg). δH (DMSO-d6, 400 MHz) 10.87 (s, 1H, NH), 8.00 (s, 1H, Ar), 7.58 (t, 1H, J=5.2, NH), 7.47 (d, 1H, J=8.8, Ar), 7.31 (d, 1H, J=8.8, Ar), 7.26 (t, 1H, J=7.2, Ar), 7.17 (d, 1H, J=7.2, Ar), 7.04 (d, 1H, J=7.2, Ar), 6.90 (t, 1H, J=7.2, Ar), 5.07 (s, 1H, CH), 4.40 (d, 2H, J=5.2, CH2), 3.88 (s, 3H, OMe), 2.43 (s, 3H, Me), 2.27 (s, 3H, Me).

### Example 172. 6-(3,5-dimethylisoxazol-4-yl)-44(2-fluorobenzyl)amino)quinolin-2(1H)-one

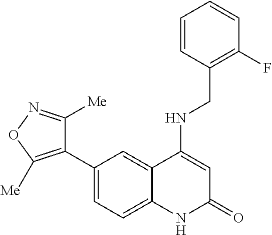 [SMILES: Cc1noc(C)c1-c1ccc2[nH]c(=O)cc(NCc3ccccc3F)c2c1]

Following a similar procedure as in Example 170, but substituting benzylamine with 2-F-benzylamine in Step 5, the desired product was obtained as a cream solid (9 mg). δH (DMSO-d6, 400 MHz) 10.92 (s, 1H, NH), 7.99 (s, 1H, Ar), 7.64 (t, 1H, J=5.6, NH), 7.47 (d, 1H, J=8.4, Ar), 7.40-7.28 (m, 3H, Ar), 7.27-7.14 (m, 2H, Ar), 5.19 (s, 1H, CH), 4.49 (d, 2H, J=5.6, CH2), 2.43 (s, 3H, Me), 2.26 (s, 3H, Me).

Examples 173-178 may be obtained in similar fashion to Example 170.

### Example 179. 4-(benzylamino)-6-(3,5-dimethylisoxazol-4-yl)-N-(2-hydroxyethyl)-2-oxo-1,2-dihydroquinoline-7-carboxamide

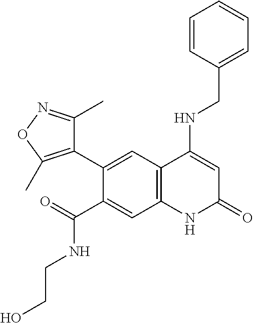 [SMILES: Cc1noc(C)c1-c1cc2c(NCc3ccccc3)cc(=O)[nH]c2cc1C(=O)NCCO]

4-(Benzylamino)-6-(3,5-dimethylisoxazol-4-yl)-N-(2-hydroxyethyl)-2-oxo-1,2-dihydroquinoline-7-carboxamide (3 mg, 8%) was obtained as a white solid following a similar procedure outlined in Example 160, Step 9, via 4-(benzylamino)-6-(3,5-dimethylisoxazol-4-yl)-2-oxo-1,2-dihydroquinoline-7-carboxylic acid (35 mg, 0.09 mmol) and 2-aminoethanol (11 mg, 0.18 mmol) by using PyBOP (66 mg, 0.13 mmol) as coupling reagent. LC-MS (ESI): m/z (M+1) 433.3. ¹H NMR (400 MHz, DMSO) δ 10.96 (s, OH), 8.20 (t, J=5.6 Hz, OH), 7.95 (s, OH), 7.69 (t, J=5.9 Hz, OH), 7.39-7.31 (m, J=8.9, 5.6 Hz, 2H), 7.28-7.24 (m, OH), 5.21 (s, OH), 4.64 (t, J=5.4 Hz, OH), 4.43 (d, J=5.6 Hz, 1H), 3.30 (s, 1H), 3.21-3.15 (m, 1H), 2.25 (s, 3H), 2.09 (s, 3H).

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A compound of Formula I, or a pharmaceutically acceptable salt thereof 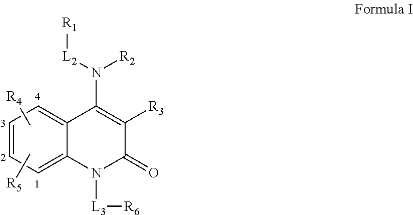
wherein:
L₂ is —N(R₇)— or —(NCOR₇)—;
R₁ is selected from the group consisting of alkyl, cycloalkyl, heterocycloalkyl, aryl and heteroaryl, wherein said heteroaryl or heterocycloalkyl include one or more nitrogen (N), oxygen (O) or sulfur (S) atoms; wherein said alkyl, cycloalkyl, heterocycloalkyl, aryl and heteroaryl are optionally substituted with one or more R₁₂;
Optionally, R₁ and R₇ may be taken together with the attached nitrogen to form a 5- to 7-membered heterocycloalkyl ring, optionally fused with an aryl group or unfused, wherein said heterocycloalkyl ring may contain an additional one or more N, O or S atoms; wherein said heterocycloalkyl ring may optionally be substituted at any position by one or more R₁₂;
R₂ is H, alkyl, —C(O)R₇, —CH₂C(O)OR₇, —OC(O)NR₇R₈, —C(O)NR₇R₈ or —C(O)OR₇, wherein said alkyl is optionally substituted with one or more R₁₂;
L₃ is a bond, —(CR₁₀R₁₁)ₙ—, —C(O)NR₁₀—, —S(O)₂NR₁₀—, —R₁₀C(O)NR₁₁—, or —OC(O)NR₁₀; and n is 0, 1, 2, or 3;
R₃ is hydrogen (H) or alkyl, wherein said alkyl is optionally substituted with one or more R₁₂;
R₄ and R₅ are independently selected for each occurrence from the group consisting of H, alkyl, halogen, CN, CF₃, NO₂, C(O)OR₇, OC(O)NR₇R₈, C(O)NR₇R₈, NR₇R₈, NR₇C(O)R₈, NR₇C(O)OR₈NR₇S(O)₂R₈, NR₇C(O)NR₈R₉, aryl, heteroaryl, cycloalkyl and heterocycloalkyl, wherein said heterocycloalkyl or heteroaryl include one or more N, O or S atoms; wherein said alkyl, aryl, heteroaryl, cycloalkyl and heterocycloalkyl are each optionally substituted with one or more R₁₂;
R₆ is selected from the group consisting of H, alkyl, aryl, heteroaryl, cycloalkyl and heterocycloalkyl, wherein said heterocycloalkyl or heteroaryl include one or more N, O or S atoms; wherein said alkyl, aryl, heteroaryl, cycloalkyl and heterocycloalkyl are each optionally substituted with one or more R₁₂;
R₇, R₈ and R₉ are independently selected for each occurrence from the group consisting of H, alkyl, heteroalkyl, aryl, arylalkyl, heteroaryl, heteroarylalkyl, cycloalkyl and heterocycloalkyl, wherein said alkyl, heteroalkyl, aryl, arylalkyl, heteroaryl, heteroarylalkyl, cycloalkyl and heterocycloalkyl are each optionally substituted with one or more R₁₂;
Optionally, R₇ and R₈, together with the included atoms, may form a 4-, 5-, 6- or 7-membered cyclic ring system, wherein said cyclic ring system is a mono or bicyclic ring optionally having an additional from one to four heteroatoms selected from N, O and S; wherein said cyclic ring system is optionally substituted with one or more of hydroxyl, sulfhydryl, alkoxy, thioalkoxy, alkyl or halogen;
Optionally, R₈ and R₉, together with the included atoms, may form a 4-, 5-, 6- or 7-membered cyclic ring system, wherein said cyclic ring system is a mono or bicyclic ring optionally having an additional from one to four heteroatoms selected from N, O and S; wherein said cyclic ring system is optionally substituted with one or more of hydroxyl, sulfhydryl, alkoxy, thioalkoxy, alkyl or halogen;
R₁₀ and R₁₁ are independently selected for each occurrence from H or alkyl, wherein said alkyl may optionally be substituted with one or more R₁₂;
and
R₁₂ is independently selected for each occurrence from the group consisting of lower (C₁-C₆) alkyl, lower alkenyl, lower alkynyl, aryl, heteroaryl, alicyclic, heterocyclic, arylalkyl, heteroarylalkyl, lower alkoxy, aryloxy, amino, alkylamino, dialkylamino, diarylalkylamino, alkylthio, arylthio, heteroarylthio, oxo, oxa, —C(O)—, —C(O)OR, —C(O)NH₂, CO₂H, acyloxy, H, halo, —CN, —NO₂, —N₃, —SH, —OH, —C(O)CH₃, perhaloalkyl, perhaloalkoxy, perhaloacyl, guanidinyl, pyridinyl, thiophene, furanyl, indolyl, indazolyl, phosphonate, phosphonic acid, phosphate, phosphoramide, sulfonate, sulfone, sulfate, sulphonamide, carbamate, urea, thiourea, thioamide and thioalkyl.

2. A compound of claim 1, or a pharmaceutically acceptable salt thereof, wherein R₁ is aryl or heteroaryl, wherein said aryl or heteroaryl is optionally substituted with one or more R₁₂.

3. A compound of claim 1, or a pharmaceutically acceptable salt thereof, wherein R₂ is hydrogen, alkyl, —C(O)R₇, or —CH₂C(O)OR₇, wherein said alkyl is optionally substituted with one or more R₁₂.

4. A compound of claim 1, or a pharmaceutically acceptable salt thereof, wherein L₂ is —NR₇, and R₁ and R₇ together with the included nitrogen form a heterocycloalkyl ring system, wherein said heterocycloalkyl is optionally substituted with one or more R₁₂;

5. A compound of claim 1, or a pharmaceutically acceptable salt thereof, wherein R₄ and R₅ are independently selected for each occurrence from the group consisting of H, aryl, heteroaryl and heterocycloalkyl, wherein said heterocycloalkyl and heteroaryl include one or more N, O or S atoms; wherein said aryl, heteroaryl and heterocycloalkyl may each optionally be substituted with one or more R₁₂;

6. A compound of claim 5, or a pharmaceutically acceptable salt thereof, wherein R₄ and R₅ are independently selected for each occurrence from the group consisting of H and heteroaryl, wherein said heteroaryl is optionally substituted with one or more R₁₂;

7. A compound of claim 1, or a pharmaceutically acceptable salt thereof, wherein L₃ is a bond or —(CR₁₀R₁₁)ₙ—.

8. A compound of claim 1, or a pharmaceutically acceptable salt thereof, wherein L₂ is —N(R₇)—, according to Formula III

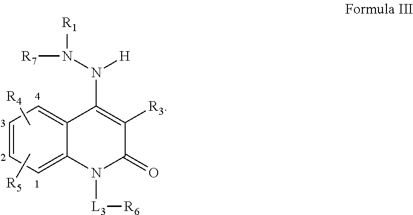

9. A compound of claim 8, selected from those compounds defined according to the following Table, or pharmaceutically acceptable salts thereof:

| | | | | Position, | | |
|---|---|---|---|---|---|---|
| R₁ | R₇ | R₃ | Position, R₄ | R₅ | L₃ | R₄ |
| 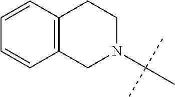 | | H | 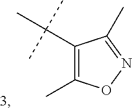 | H | bond | H |
| 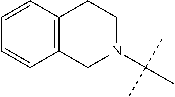 | | CH₃ | 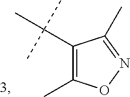 | H | bond | H |
| 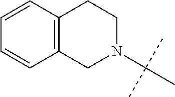 | | H | H | 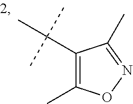 | bond | H |
| 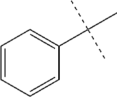 | H | H | 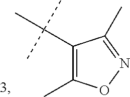 | H | bond | H |
| 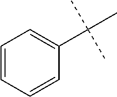 | CH₃ | H | 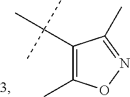 | H | bond | H |
| 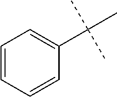 | CH₂COOH | H | 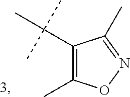 | H | bond | H |
| 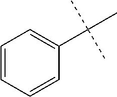 | COCH₃ | H | 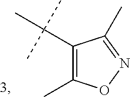 | H | bond | H |
| 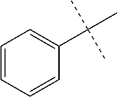 | H | H | 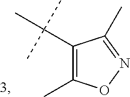 | H | —CH2— | H |
| 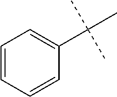 | CH₃ | H | 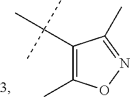 | H | —CH2— | H |
| 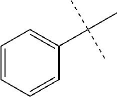 | CH₂COOH | H | 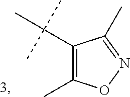 | H | —CH2— | H |
| 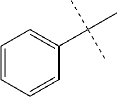 | COCH₃ | H | 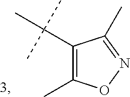 | H | —CH2— | H |
| 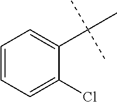 | H | H | 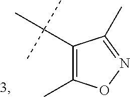 | H | bond | H |
| 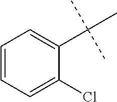 | CH₃ | H | 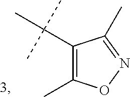 | H | bond | H |
| 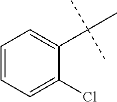 | CH₂COOH | H | 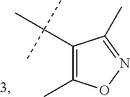 | H | bond | H |
| 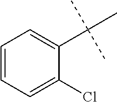 | COCH₃ | H | 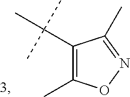 | H | bond | H |
| 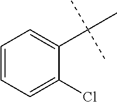 | H | H | 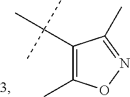 | H | —CH2— | H |
| 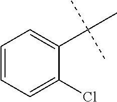 | CH₃ | H | 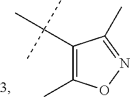 | H | —CH2— | H |
| 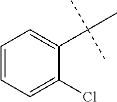 | CH₂COOH | H | 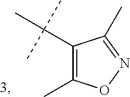 | H | —CH2— | H |
| 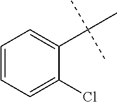 | COCH₃ | H | 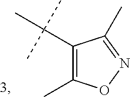 | H | —CH2— | H |
| 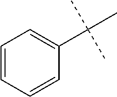 | H | H | 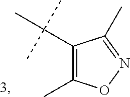 | H | bond | H |
| 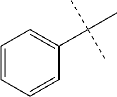 | CH₃ | CH₃ | 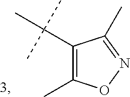 | H | bond | H |
| 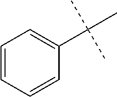 | CH₂COOH | CH₃ | 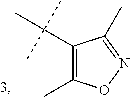 | H | bond | H |
| 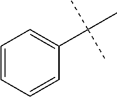 | COCH₃ | CH₃ | 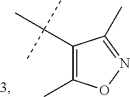 | H | bond | H |
| 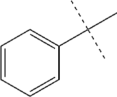 | H | CH₃ | 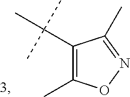 | H | —CH2— | H |
| 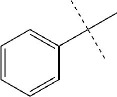 | CH₃ | CH₃ | 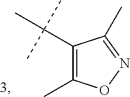 | H | —CH2— | H |
| 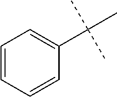 | CH₂COOH | CH₃ | 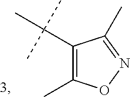 | H | —CH2— | H |
| 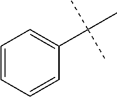 | COCH₃ | CH₃ | 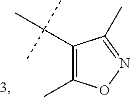 | H | —CH2— | H |
| 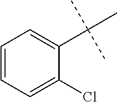 | H | CH₃ | 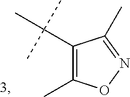 | H | bond | H |
| 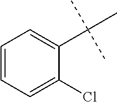 | CH₃ | CH₃ | 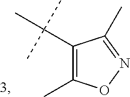 | H | bond | H |
| 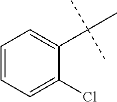 | CH₂COOH | CH₃ | 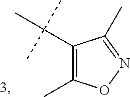 | H | bond | H |
| 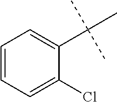 | COCH₃ | CH₃ | 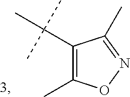 | H | bond | H |
| 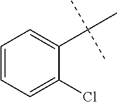 | H | CH₃ | 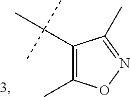 | H | —CH2— | H |
| 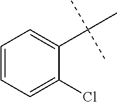 | CH₃ | CH₃ | 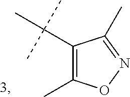 | H | —CH2— | H |
| 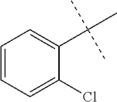 | CH₂COOH | CH₃ | 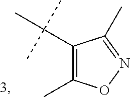 | H | —CH2— | H |
| 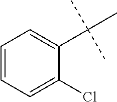 | COCH₃ | CH₃ | 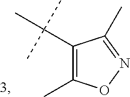 | H | —CH2— | H |
| 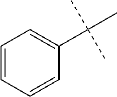 | H | H | 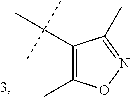 | 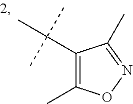 | bond | H |
| 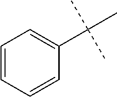 | CH₃ | H | 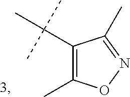 | 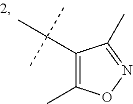 | bond | H |
| 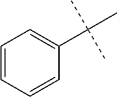 | CH₂COOH | H | 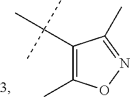 | 2, Ph | bond | H |
| 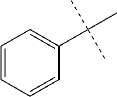 | COCH₃ | H | 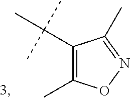 | 2, Ph | bond | H |
| 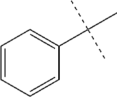 | H | H | 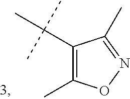 | 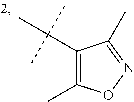 | —CH2— | H |
| 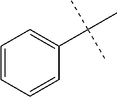 | CH₃ | H | 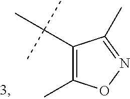 | 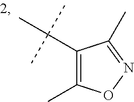 | —CH2— | H |
| 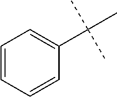 | CH₂COOH | H | 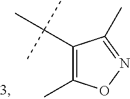 | 2, Ph | —CH2— | H |
| 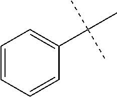 | COCH₃ | H | 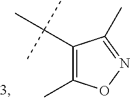 | 2, Ph | —CH2— | H |
| 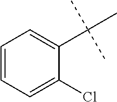 | H | H | 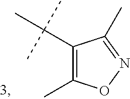 | 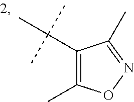 | bond | H |
| 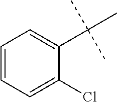 | CH₃ | H | 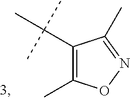 | 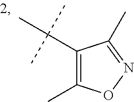 | bond | H |
| 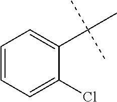 | CH₂COOH | H | 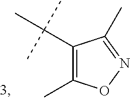 | 2, Ph | bond | H |
| 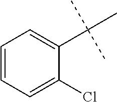 | COCH₃ | H | 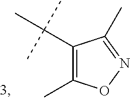 | 2, Ph | bond | H |
| 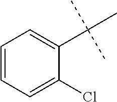 | H | H | 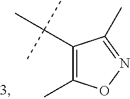 | 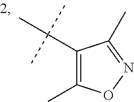 | —CH2— | H |
| 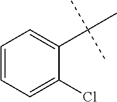 | CH₃ | H | 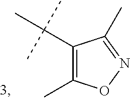 | 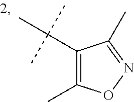 | —CH2— | H |
| 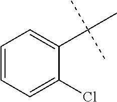 | CH₂COOH | H | 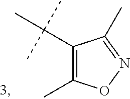 | 2, Ph | —CH2— | H |
| 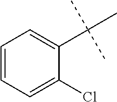 | COCH₃ | H | 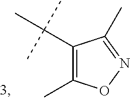 | 2, Ph | —CH2— | H |
| 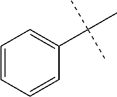 | H | H | 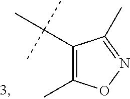 | 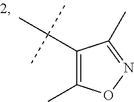 | bond | H |
| 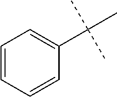 | CH₃ | CH₃ | 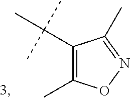 | 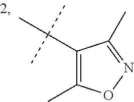 | bond | H |
| 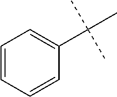 | CH₂COOH | CH₃ | 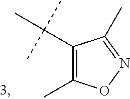 | 2, Ph | bond | H |
| 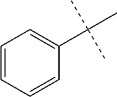 | COCH₃ | CH₃ | 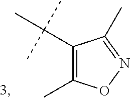 | 2, Ph | bond | H |
| 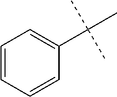 | H | CH₃ | 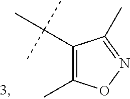 | 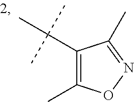 | —CH2— | H |
| 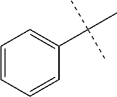 | CH₃ | CH₃ | 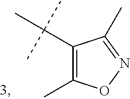 | 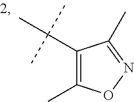 | —CH2— | H |
| 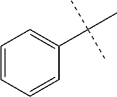 | CH₂COOH | CH₃ | 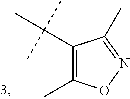 | 2, Ph | —CH2— | H |
| 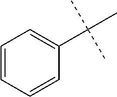 | COCH₃ | CH₃ | 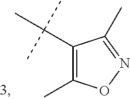 | 2, Ph | —CH2— | H |
| 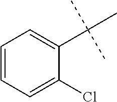 | H | CH₃ | 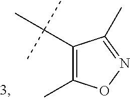 | 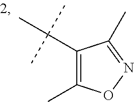 | bond | H |
| 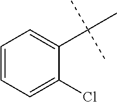 | CH₃ | CH₃ | 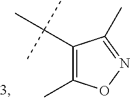 | 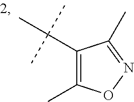 | bond | H |
| 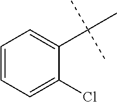 | CH₂COOH | CH₃ | 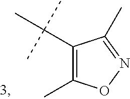 | 2, Ph | bond | H |
| 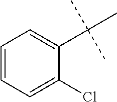 | COCH₃ | CH₃ | 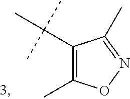 | 2, Ph | bond | H |
| 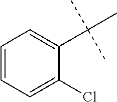 | H | CH₃ | 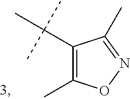 | 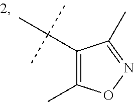 | —CH2— | H |
| 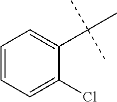 | CH₃ | CH₃ | 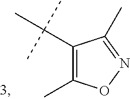 | 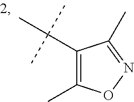 | —CH2— | H |
| 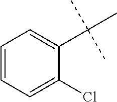 | CH₂COOH | CH₃ | 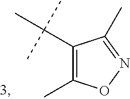 | 2, Ph | —CH2— | H |
| 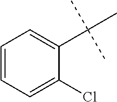 | COCH₃ | CH₃ | 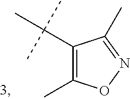 | 2, Ph | —CH2— | H |
| 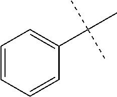 | H | H | 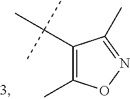 | H | —CH2— | Ph |
| 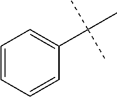 | CH₃ | H | 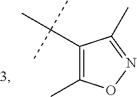 | H | —CH2— | Ph |
| 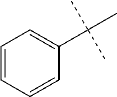 | CH₂COOH | H | 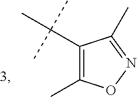 | H | —CH2— | Ph |
| 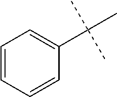 | COCH₃ | H | 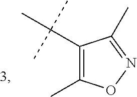 | H | —CH2— | Ph |
| 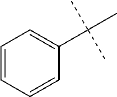 | H | H | 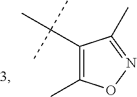 | H | —CH2— | Ph |
| 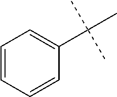 | CH₃ | H | 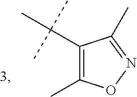 | H | —CH2— | Ph |
| 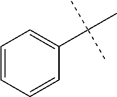 | CH₂COOH | H | 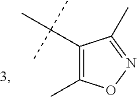 | H | —CH2— | Ph |
| 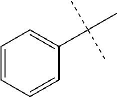 | COCH₃ | H | 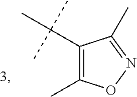 | H | —CH2— | Ph |
| 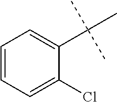 | H | H | 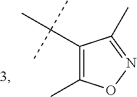 | H | —CH2— | Ph |
| 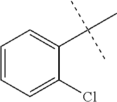 | CH₃ | H | 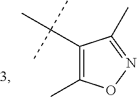 | H | —CH2— | Ph |
| 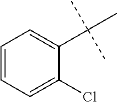 | CH₂COOH | H | 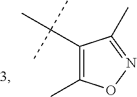 | H | —CH2— | Ph |
| 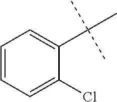 | COCH₃ | H | 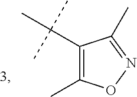 | H | —CH2— | Ph |
| 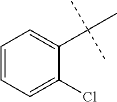 | H | H | 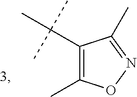 | H | —CH2— | Ph |
| 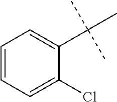 | CH₃ | H | 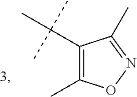 | H | —CH2— | Ph |
| 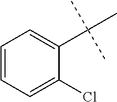 | CH₂COOH | H | 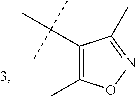 | H | —CH2— | Ph |
| 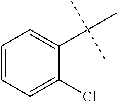 | COCH₃ | H | 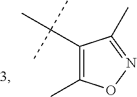 | H | —CH2— | Ph |
| 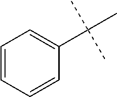 | H | H | 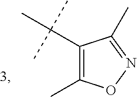 | H | —CH2— | Ph |
| 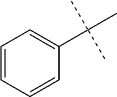 | CH₃ | CH₃ | 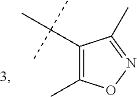 | H | —CH2— | Ph |
| 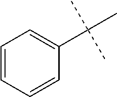 | CH₂COOH | CH3 | 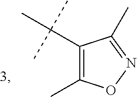 | H | —CH2— | Ph |
| 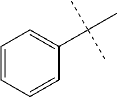 | COCH₃ | CH₃ | 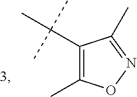 | H | —CH2— | Ph |
| 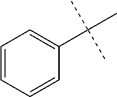 | H | CH₃ | 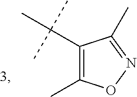 | H | —CH2— | Ph |
| 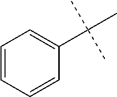 | CH₃ | CH₃ | 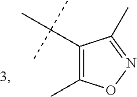 | H | —CH2— | Ph |
| 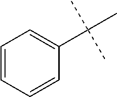 | CH₂COOH | CH₃ | 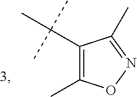 | H | —CH2— | Ph |
| 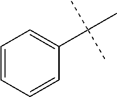 | COCH₃ | CH₃ | 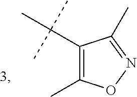 | H | —CH2— | Ph |
| 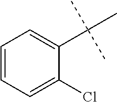 | H | CH₃ | 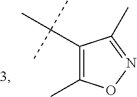 | H | —CH2— | Ph |
| 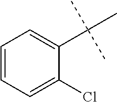 | CH₃ | CH₃ | 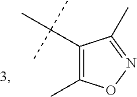 | H | —CH2— | Ph |
| 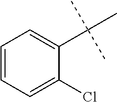 | CH₂COOH | CH₃ | 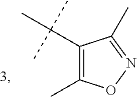 | H | —CH2— | Ph |
| 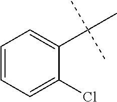 | COCH₃ | CH₃ | 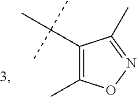 | H | —CH2— | Ph |
| 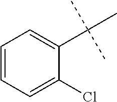 | H | CH₃ | 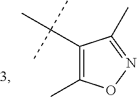 | H | —CH2— | Ph |
| 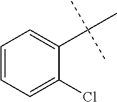 | CH₃ | CH₃ | 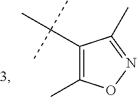 | H | —CH2— | Ph |
| 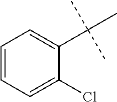 | CH₂COOH | CH₃ | 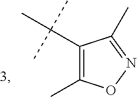 | H | —CH2— | Ph |
| 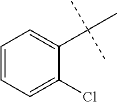 | COCH₃ | CH₃ | 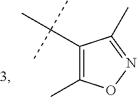 | H | —CH2— | Ph |
| 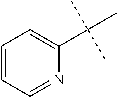 | H | H | 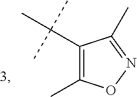 | H | —CH2— | Ph |
| 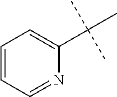 | CH₃ | H | 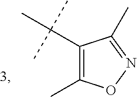 | H | —CH2— | Ph |
| 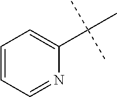 | CH₂COOH | H | 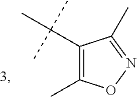 | H | —CH2— | H |
| 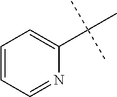 | COCH₃ | H | 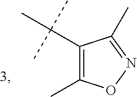 | H | —CH2— | H |
| 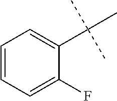 | H | H | 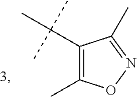 | H | bond | H |
| 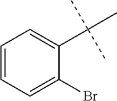 | H | H | 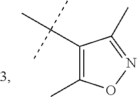 | H | bond | H |
| 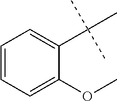 | H | H | 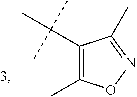 | H | bond | H |
| 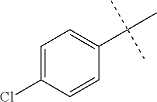 | H | H | 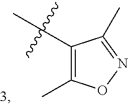 [SMILES: *c1c(C)noc1C] | H | bond | H. |

10. A compound of Formula II, or pharmaceutically acceptable salt thereof 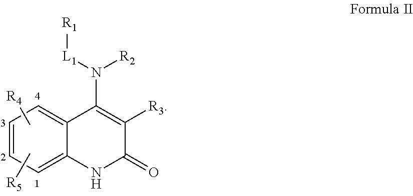
wherein:
L₁ is a bond or is —(CR₁₀R₁₁)ₘ—; and m is 0, 1, 2, or 3;
R₁ is selected from the group consisting of alkyl, cycloalkyl, heterocycloalkyl, aryl and heteroaryl, wherein each of said heterocycloalkyl and heteroaryl include one or more N, O or S atoms; wherein each of said alkyl, cycloalkyl, heterocycloalkyl, aryl and heteroaryl are optionally substituted with one or more R₁₂;
R₂ is H, alkyl, —C(O)R₇, —CH₂C(O)OR₇, —OC(O)NR₇R₈, —C(O)NR₇R₈ or —C(O)OR₇, wherein said alkyl is optionally substituted with one or more R₁₂;
R₃ is H or alkyl, wherein said alkyl is optionally substituted with one or more R₁₂;
R₄ and R₅ are independently selected for each occurrence from the group consisting of H, alkyl, halogen, CN, CF₃, NO₂, C(O)OR₇, OC(O)NR₇R₈, C(O)NR₇R₈, NR₇R₈, NR₇C(O)R₈, NR₇C(O)OR₈NR₇S(O)₂R₈, NR₇C(O)NR₈R₉, aryl, heteroaryl, cycloalkyl and heterocycloalkyl, wherein said heterocycloalkyl and heteroaryl include one or more N, O or S atoms; wherein each of said alkyl, aryl, heteroaryl, cycloalkyl or heterocycloalkyl may optionally be substituted with one or more R₁₂;
R₇, R₈ and R₉ are independently selected for each occurrence from the group consisting of H, alkyl, heteroalkyl, aryl, arylalkyl, heteroaryl, heteroarylalkyl, cycloalkyl and heterocycloalkyl, wherein each of said alkyl, heteroalkyl, aryl, arylalkyl, heteroaryl, heteroarylalkyl, cycloalkyl and heterocycloalkyl groups may optionally be substituted with one or more R₁₂;
Optionally, R₇ and R₈, together with the included atoms, may form a 4-, 5-, 6- or 7-membered cyclic ring system, wherein said cyclic ring system is a mono or bicyclic ring optionally having an additional from one to four heteroatoms selected from N, O and S; wherein said cyclic ring system is optionally be substituted with one or more R₁₂;
Optionally, R₈ and R₉, together with the included atoms, may form a 4-, 5-, 6- or 7-membered cyclic ring system, wherein said cyclic ring system is a mono or bicyclic ring optionally having an additional from one to four heteroatoms selected from N, O and S; wherein said cyclic ring system is optionally be substituted with one or more R₁₂;
R₁₀ and R₁₁ are independently chosen for each occurrence from H or alkyl, wherein said alkyl may optionally be substituted with one or more R₁₂;
and
R₁₂ is independently selected for each occurrence from the group consisting of lower (C₁-C₆) alkyl, lower alkenyl, lower alkynyl, aryl, heteroaryl, alicyclic, heterocyclic, arylalkyl, heteroarylalkyl, lower alkoxy, aryloxy, amino, alkylamino, dialkylamino, diarylalkylamino, alkylthio, arylthio, heteroarylthio, oxo, oxa, —C(O)—, —C(O)OR, —C(O)NH₂, CO₂H, acyloxy, H, halo, —CN, —NO₂, —N₃, —SH, —OH, —C(O)CH₃, perhaloalkyl, perhaloalkoxy, perhaloacyl, guanidinyl, pyridinyl, thiophene, furanyl, indolyl, indazolyl, phosphonate, phosphonic acid, phosphate, phosphoramide, sulfonate, sulfone, sulfate, sulphonamide, carbamate, urea, thiourea, thioamide and thioalkyl.

11. A compound of claim 10, or a pharmaceutically acceptable salt thereof, wherein R₁ is aryl, heteroaryl, cycloalkyl or heterocycloalkyl, wherein the said heteroaryl and heterocycloalkyl include one or more N, O or S atoms; wherein each said aryl, heteroaryl, cycloalkyl and heterocycloalkyl may optionally be substituted with one or more R₁₂.

12. A compound of claim 10, or a pharmaceutically acceptable salt thereof, wherein R₂ is H or alkyl, wherein said alkyl is optionally substituted with one or more R₁₂.

13. A compound of claim 10, or a pharmaceutically acceptable salt thereof, wherein R₃ is H.

14. A compound of claim 10, or a pharmaceutically acceptable salt thereof, wherein R₄ and R₅ are independently selected from the group consisting of alkyl, aryl, heteroaryl, —C(O)NH₂ and —C(O)NH(CH₂)ₖOH, wherein k is 2 or 3; wherein each of said alkyl, aryl and heteroaryl is optionally substituted with one or more R₁₂.

15. A compound of claim 10, selected from those compounds defined according to the following Table, or pharmaceutically acceptable salts thereof:

| R₁ | L₁ | R₂ | R₃ | Position, R₄ | Position, R₅ |
|---|---|---|---|---|---|
| 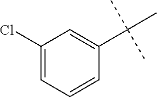 | —CH2— | H | H | 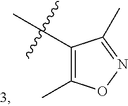 [SMILES: *c1c(C)noc1C] | H |
| 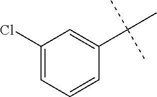 | —CH2— | CH₃ | H | 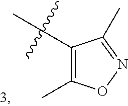 [SMILES: *c1c(C)noc1C] | H |
| 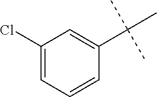 | (R); —CH(CH₃)— | H | H | 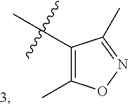 [SMILES: *c1c(C)noc1C] | H |
| 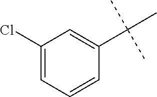 | (S); —CH(CH₃)— | CH₃ | H | 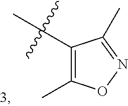 [SMILES: *c1c(C)noc1C] | H |
| 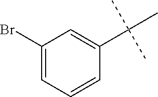 | —CH2— | H | H | 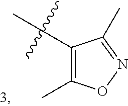 [SMILES: *c1c(C)noc1C] | H |
| 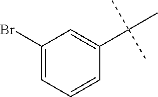 | —CH2— | CH₃ | H | 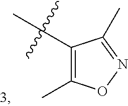 [SMILES: *c1c(C)noc1C] | H |
| 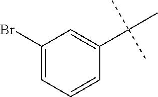 | (R); —CH(CH₃)— | H | H | 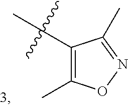 [SMILES: *c1c(C)noc1C] | H |
| 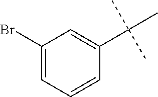 | (S); —CH(CH₃)— | CH₃ | H | 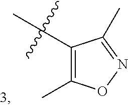 [SMILES: *c1c(C)noc1C] | H |
| 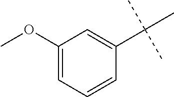 | —CH2— | H | H | 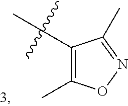 [SMILES: *c1c(C)noc1C] | H |
| 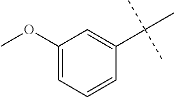 | —CH2— | CH₃ | H | 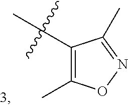 [SMILES: *c1c(C)noc1C] | H |
| 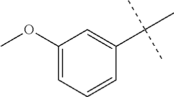 | (R); —CH(CH₃)— | H | H | 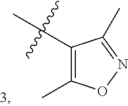 [SMILES: *c1c(C)noc1C] | H |
| 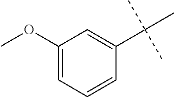 | (S); —CH(CH₃)— | CH₃ | H | 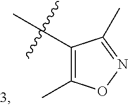 [SMILES: *c1c(C)noc1C] | H |
| 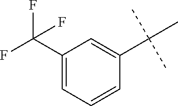 | —CH2— | H | H | 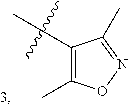 [SMILES: *c1c(C)noc1C] | H |
| 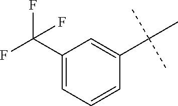 | —CH2— | CH₃ | H | 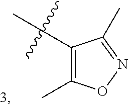 [SMILES: *c1c(C)noc1C] | H |
| 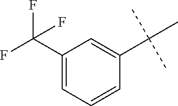 | (R); —CH(CH₃)— | H | H | 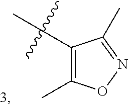 [SMILES: *c1c(C)noc1C] | H |
| 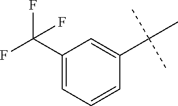 | (S); —CH(CH₃)— | CH₃ | H | 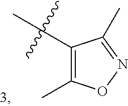 [SMILES: *c1c(C)noc1C] | H |
| 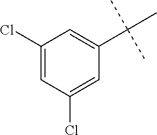 | —CH2— | H | H | 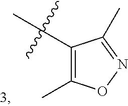 [SMILES: *c1c(C)noc1C] | H |
| 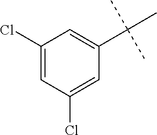 | —CH2— | CH₃ | H | 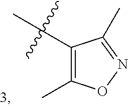 [SMILES: *c1c(C)noc1C] | H |
| 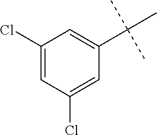 | (R); —CH(CH₃)— | H | H | 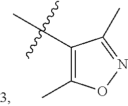 [SMILES: *c1c(C)noc1C] | H |
| 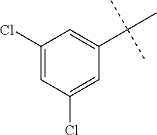 | (S); —CH(CH₃)— | CH₃ | H | 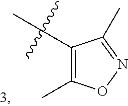 [SMILES: *c1c(C)noc1C] | H |
| 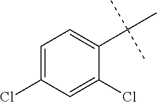 | —CH2— | H | H | 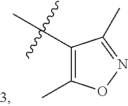 [SMILES: *c1c(C)noc1C] | H |
| 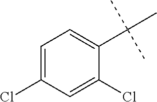 | —CH2— | CH₃ | H | 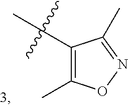 [SMILES: *c1c(C)noc1C] | H |
| 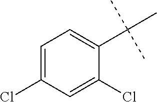 | (R); —CH(CH₃)— | H | H | 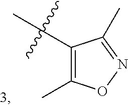 [SMILES: *c1c(C)noc1C] | H |
| 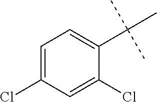 | (S); —CH(CH₃)— | CH₃ | H | 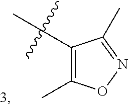 [SMILES: *c1c(C)noc1C] | H |
| 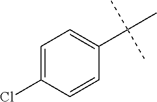 | —CH2— | H | H | 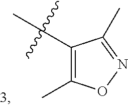 [SMILES: *c1c(C)noc1C] | H |
| 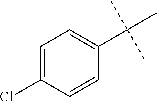 | —CH2— | CH₃ | H | 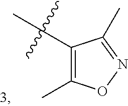 [SMILES: *c1c(C)noc1C] | H |
| 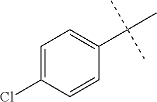 | (R); —CH(CH₃)— | H | H | 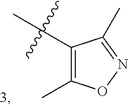 [SMILES: *c1c(C)noc1C] | H |
| 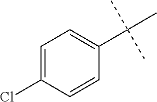 | (S); —CH(CH₃)— | CH₃ | H | 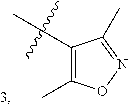 [SMILES: *c1c(C)noc1C] | H |
| 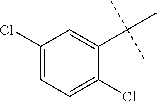 | —CH2— | H | H | 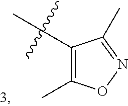 [SMILES: *c1c(C)noc1C] | H |
| 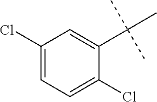 | —CH2— | CH₃ | H | 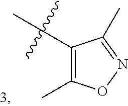 [SMILES: *c1c(C)noc1C] | H |
| 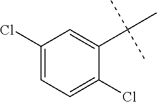 | (R); —CH(CH₃)— | H | H | 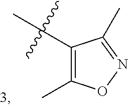 [SMILES: *c1c(C)noc1C] | H |
| 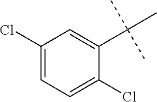 | (S); —CH(CH₃)— | CH₃ | H | 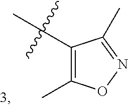 [SMILES: *c1c(C)noc1C] | H |
| 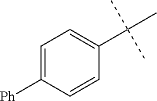 | —CH2— | H | H | 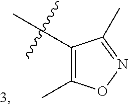 [SMILES: *c1c(C)noc1C] | H |
| 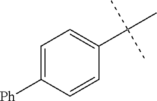 | —CH2— | CH₃ | H | 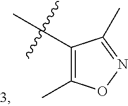 [SMILES: *c1c(C)noc1C] | H |
| 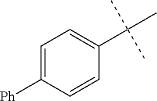 | (R); —CH(CH₃)— | H | H | 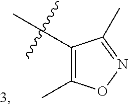 [SMILES: *c1c(C)noc1C] | H |
| 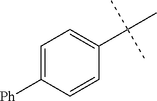 | (S); —CH(CH₃)— | CH₃ | H | 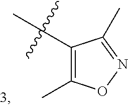 [SMILES: *c1c(C)noc1C] | 2, Ph |
| 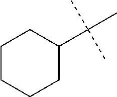 | —CH2— | H | H | 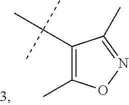 | H |
| 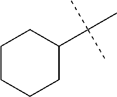 | —CH2— | CH₃ | H | 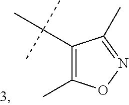 | H |
| 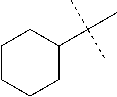 | (R); —CH(CH₃)— | H | H | 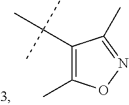 | H |
| 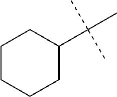 | (S); —CH(CH₃)— | CH₃ | H | 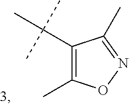 | H |
| 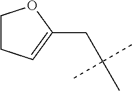 | —CH2— | H | H | 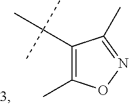 | H |
| 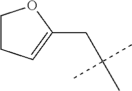 | —CH2— | CH₃ | H | 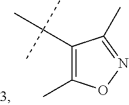 | H |
| 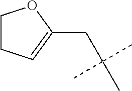 | (R); —CH(CH₃)— | H | H | 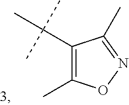 | 2, CH₃ |
| 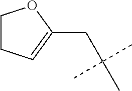 | (S); —CH(CH₃)— | CH₃ | H | 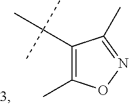 | 2, CH₃ |
| 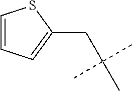 | —CH2— | H | H | 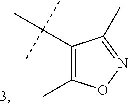 | H |
| 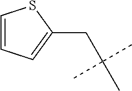 | —CH2— | CH₃ | H | 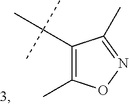 | H |
| 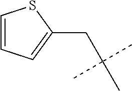 | (R); —CH(CH₃)— | H | H | 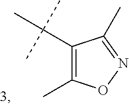 | H |
| 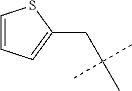 | (S); —CH(CH₃)— | CH₃ | H | 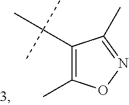 | H |
| 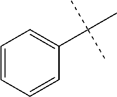 | (R); —C(H)(CH₂CH₃)— | H | H | 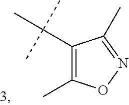 | H |
| 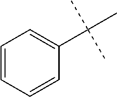 | (S); —C(H)(CH₂CH₃)— | CH₃ | H | 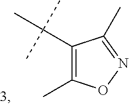 | H |
| 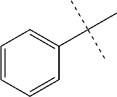 | (R); —C(H)(CH₂CH₃)— | H | H | 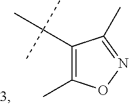 | H |
| 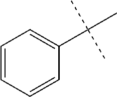 | (S); —C(H)(CH₂CH₃)— | CH₃ | H | 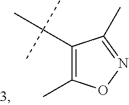 | H |
| 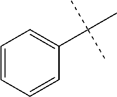 | —CH2— | H | H | 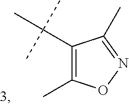 | 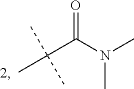 |
| 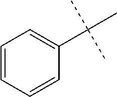 | —CH2— | H | H | 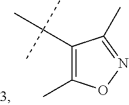 | 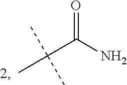 |
| 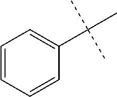 | —CH2— | H | H | 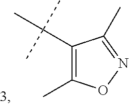 | 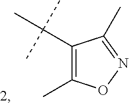 |
| 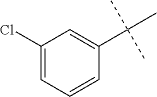 | —CH2— | H | H | 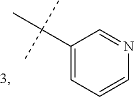 | H |
| 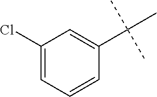 | —CH2— | H | H | 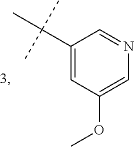 | H |
| 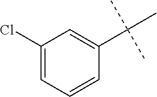 | —CH2— | H | H | 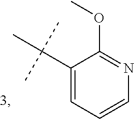 | H |
| 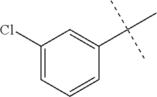 | —CH2— | H | H | 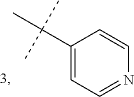 | H |
| 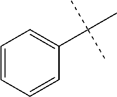 | —CH2— | H | H | 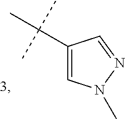 | H |
| 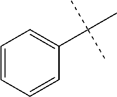 | —CH2— | H | H | 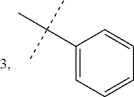 | H |
| 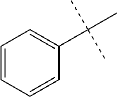 | —CH2— | H | H | 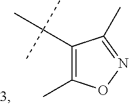 | 1, F |
| 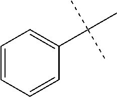 | —CH2— | H | H | 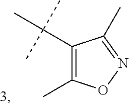 | H |
| 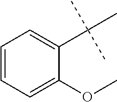 | —CH2— | H | H | 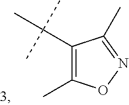 | H |
| 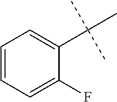 | —CH2— | H | H | 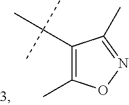 | H |
| 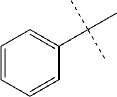 | (R); —CH(CH₃)— | H | H | 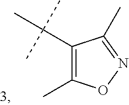 | H |
| 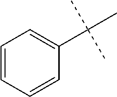 | (S); —CH(CH₃)— | CH₃ | H | 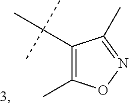 | H |
| 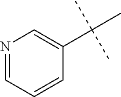 | —CH2— | H | H | 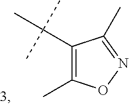 | H |
| 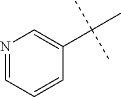 | —CH2— | CH₃ | H | 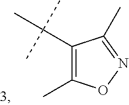 | H |
| 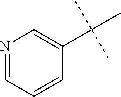 | (R); —CH(CH₃)— | H | H | 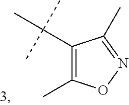 | H |
| 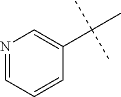 | (S); —CH(CH₃)— | CH₃ | H | 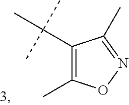 | H |
| 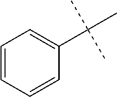 | —CH2— | H | H | 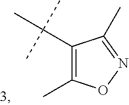 | 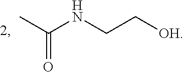 |

16. A pharmaceutical composition comprising a compound of claim 1, or a pharmaceutically acceptable salt thereof, and a pharmaceutically acceptable carrier.

17-18. (canceled)

19. A method for the treatment or prevention of a disease selected from the group consisting of human NUT midline carcinoma, multiple myeloma, Burkitt's lymphoma, myeloid leukemia, NPM1c mutant leukemia, T-cell lymphoblastic leukemia, hepatocellular carcinoma, glioblastoma, neuroblastoma, sarcoma, breast cancer, colorectal cancer, lung cancer, pancreatic cancer, neuroendocrine tumors, Merkel cell carcinoma, prostate cancer, osteoarthritis, rheumatoid arthritis, Alzheimer's disease, and HIV infection, in a subject, comprising administering to the subject a therapeutically effective amount of a compound of claim 1, or a pharmaceutically acceptable salt thereof.

20-28. (canceled)

29. A pharmaceutical composition comprising a compound of claim 10, or a pharmaceutically acceptable salt thereof, and a pharmaceutically acceptable carrier.

30. A method for the treatment or prevention of a disease selected from the group consisting of human NUT midline carcinoma, multiple myeloma, Burkitt's lymphoma, myeloid leukemia, NPM1c mutant leukemia, T-cell lymphoblastic leukemia, hepatocellular carcinoma, glioblastoma, neuroblastoma, sarcoma, breast cancer, colorectal cancer, lung cancer, pancreatic cancer, neuroendocrine tumors, Merkel cell carcinoma, prostate cancer, osteoarthritis, rheumatoid arthritis, Alzheimer's disease, and HIV infection, in a subject, comprising administering to the subject a therapeutically effective amount of a compound of claim 10, or a pharmaceutically acceptable salt thereof.
